(19) United States
(12) Patent Application Publication
(10) Pub. No.: US 2018/0216133 A1
(43) Pub. Date: Aug. 2, 2018

(54) COMPOSITIONS AND METHODS FOR ACHIEVING HIGH LEVELS OF TRANSDUCTION IN HUMAN LIVER CELLS

(71) Applicant: The Trustees of the University of Pennsylvania, Philadelphia, PA (US)

(72) Inventors: James M. Wilson, Philadelphia, PA (US); Lili Wang, Phoenixville, PA (US); Qiang Wang, Philadelphia, PA (US)

(21) Appl. No.: 15/745,379

(22) PCT Filed: Jul. 15, 2016
(86) PCT No.: PCT/US16/42472

§ 371 Date: Jan. 16, 2018

Related U.S. Application Data
(60) Provisional application No. 62/193,621, filed on Jul. 17, 2015.

(51) Int. Cl. C12N 15/86; C12N 7/00; A61K 48/00; A61K 35/761

(52) U.S. Cl. CPC C12N 15/86; C12N 7/00; A61K 48/0058; C12N 2750/14151; C12N 2750/14122; C12N 2750/14143; C12N 2750/14145; A61K 35/761

(57) ABSTRACT

Use of a rAAV3B vector to deliver gene products to human hepatocytes is described. The rAAV3B vectors achieve high levels of transduction even in the presence of pre-existing immunity to AAV8 or AAVrh10. Compositions and treatment regimens are described. Also provided are rAAV engineered to facilitate purification and methods of purifying the AAV.

### STATEMENT OF FEDERALLY SPONSORED RESEARCH

This work was supported by National Institutes of Health grants: NICHD P01-HD057247, NHLBI P01-HL059407, and NIDDK P30-DK047757. The US government may have certain rights in this invention.

### BACKGROUND OF THE INVENTION

Liver is the desired target for gene transfer in the treatment of a variety of inherited diseases. A number of viral and non-viral vectors have been evaluated for liver-directed gene therapy although it has been reported that vectors based on adeno-associated viruses (AAV) show significant promise [Hastie, E. and Samulski R J, Hum Gene Ther, 26: 257-265 (2015)]. Initial work utilized vectors based on AAV serotype 2 to treat hemophilia B [Snyder, et al, Nat Genet, 16: 270-276 (1997); Wang, L., et al, Proc Natl Acad Sci, 96: 3906-3910 (1999); Nathwani A C, et al., Blood, 100: 1662-1669 (2002); Mount, J D, et al, Blood, 99: 2670-2676 (2002)]. However, in clinical trials, transduction of hepatocytes was low and transgene expression was transient [Manno, C S, et al, Nat Med, 122: 342-347 (2006)].

Initial attempts to improve performance of AAV2 vectors used capsids from the other five capsid serotypes that had been isolated in the 1960s as contaminants of primate adenoviruses [Mingozzi F., et al, J Virol., 76: 10497-10502 (2002); Grimm, D, et al, Blood, 102: 2412-2419]. The results were mixed with some capsids demonstrating improved transduction of tissues other than liver. Vectors based on AAV1 showed improved transduction of skeletal and cardiac muscle forming the basis of a commercially approved product (i.e., Glybera) [Xiao, W. et al, J. Virol., 73: 3994-4003 (1999); Yla-Herttuala, S, Mol Ther, 20: 1831-1932 (2012)]. The utility of AAV vectors was expanded by the Wilson Laboratory through the isolation of over 100 natural capsid variants from human and non-human primates, some of which showed substantial improvements in targeting liver [Gao, G P, et al, Proc Natl Acad Sci USA, 99: 11854-11859 (2002)]. A systematic evaluation of these novel capsids in mice and nonhuman primates identified AAV8 as the preferred capsid for liver directed gene therapy [Wang, L., et al, Mol Ther, 18: 118-125 (2012); Wang, L., et al, Mol Ther, 18: 126-134 (2010)]. Hemophilia B patients treated with an AAV8 vector showed dose dependent expression of factor IX that has been stable for at least 4-5 years; this has reduced and in some cases eliminated the requirement for protein replacement [Nathwani, A C, et al, N Engl J Med, 365: 2357-2365 (2011); Nathwani, A C, et al, N Engl. J Med, 371: 1994-2004 (2014)]. Two other capsids isolated from primate tissues, AAVrh10 and AAV9, are in the clinic for treating several neurodegenerative diseases [NCT01161576. Safety Study of a Gene Transfer Vector (AAVrh10) for Children With Late Infantile Neuronal Ceroid Lipofuscinosis. ClinicalTrials.gov.; 1 NCT01414985. AAVRh10 Administered to Children With Late Infantile Neuronal Ceroid Lipofuscinosis With Uncommon Genotypes or Moderate/Severe Impairment. Clinicaltrials.gov.; NCT02122952. Gene Transfer Clinical Trial for Spinal Muscular Atrophy Type 1. ClinicalTrials.gov].

More recent attempts to improve vector performance have used existing AAV capsids to create diverse populations of engineered variants which are propagated under selective pressure to isolate those with the desired property which in most cases is improved transduction [Mahreshri, et al, Nat biotechnol, 24: 198-204 (2006); Perabo, L., et al, J Gene Med, 8: 155-162 (2006); Koerber, J T, et al, Nat Protoc, 1: 701-706; Grimm, et al, J Virol, 82: 5887-5911 (2008); Li, W., et al, Mol Ther, 16: 1252-1260; Koerber, J T, et al, Mol Ther, 16: 1703-1709 (2008); Lisowski, L, et al, Nature, 506: 382-386 (2014)]. The challenge is establishing a selection system that recapitulates in vivo delivery in humans.

Liver directed gene therapy has advanced into the clinic on multiple fronts for hemophilia B [Mann, C S, et al, Nat Med, 12: 342-347 (2006); Nathwani, A C, et al, N Engl J Med, 365: 2357-2365 (2011); Nathwani, A C, N Engl J Med, 371: 1994-2004 (2014)]. The first clinical trial used AAV2 which did not progress beyond phase I due to low level and transient expression of factor IX concurrent with liver toxicity [Marino, C S, et al, 2006, cited above]. Based on promising pre-clinical studies in mice and monkeys, several groups conducted clinical trials with AAV8 based vectors in patients with hemophilia B. The St. Jude's/UCL trial achieved low level but stable expression of factor IX without dose limiting toxicities that has substantially reduced or eliminated the need for traditional protein replacement treatments [Nathwani, A C, et al, N Engl J Med., 365: 2357-2365 (2011); Nathwani, A C, et al, N Engl J Med, 371: 1994-2004 (2014)]. These seminal human proof-of-concept studies bode well for the use of AAV8 vectors in other liver based diseases.

The engineered capsid AAVLK03 was isolated by Lisowski et al following DNA shuffling and selection in the human liver xenograft model [Lisowski, L, et al, Nature, 506: 382-386 (2014)]. They assert that AAVLK03 vectors are substantially more efficient than AAV8 and AAV3B vectors for human liver gene therapy based on studies in the human liver xenograft model. However, Srivastava and co-workers have shown high transduction of human hepatoma cell lines with vectors based on AAV3B [Cheng, B., et al, Gene Ther, 19: 375-384 (2012); Ling, et al, Hum Gene Ther, 25: 1023-1034 (2014)].

What are needed are next generation AAV vectors for liver gene therapy which have good transduction efficiency and which are serologically distinct from AAV8.

### SUMMARY OF THE INVENTION

In one aspect, a regimen for delivery of a gene product to a human patient is provided. The regimen comprises (a) delivery of a first recombinant AAV vector comprising an expression cassette comprising an exogenous sequence encoding a gene product under control of regulatory sequences which direct expression thereof in a cell; and (b) delivery of a second recombinant AAV vector comprising an expression cassette comprising an exogenous sequence encoding a gene product under control of regulatory sequences which direct expression of the product in a cell, wherein the first recombinant AAV vector or the second AAV vector has an AAV3B capsid. In one embodiment, the other of the first or the second AAV vector has a capsid which is selected from AAV8, AAV2 or rh10.

In a further aspect, the invention involves targeting hepatocytes of the patient.

In one aspect, the delivery of the first rAAV and the second rAAV are temporally separated by at least about one month, at least about three months, or about 1 year to about 10 years.

In a further aspect, the regimen further comprises delivery of at least a third AAV, wherein said third AAV has a capsid which differs from AAV3B.

In yet another aspect, a method is provided for targeting human spleen cells which comprises delivering a recombinant AAV vector comprising an AAV3B capsid having packaged therein an expression cassette comprising an exogenous sequence encoding a gene product under control of regulatory sequences which direct expression thereof in a cell.

In still another aspect, a method is provided for targeting human hepatocytes in a patient having pre-existing immunity to AAV8 or AAVrh10, said method comprising delivering a recombinant AAV vector comprising an AAV3B capsid having packaged therein an expression cassette comprising an exogenous sequence encoding a gene product under control of regulatory sequences which direct expression thereof in a cell.

Further, a method is provided for providing high hepatocyte transduction levels in vivo in a human patient having pre-existing immunity to a clade E AAV. The method involves delivering a recombinant AAV vector comprising an AAV3B capsid having packaged therein an expression cassette comprising an exogenous sequence encoding a gene product under control of regulatory sequences which direct expression thereof in a cell.

In another embodiment, methods for delivering genes via rAAV having engineered capsids which have at least one engineered affinity column binding epitope is provided. Further described are methods for purifying such engineered rAAV. Also described is the use of such engineered rAAV for delivery of a gene to a target host cell. In one embodiment, the engineered purification epitope does not significantly alter transduction efficiency and/or tropism (i.e., the target cell population).

Still other aspects and advantages of the invention will be apparent from the following detailed description of the invention.

### BRIEF DESCRIPTION OF THE DRAWINGS

FIG. 1 shows transduction efficiency (GFP) in mouse liver (2 weeks post injection). C57BL/6 male mice received intravenous injection of 1×10¹¹ GC of AAV3B, LK03.L125I, LK03, rh10, AAV8 or AAV2.TBG.GFP vector or 3×10″ GC of AAV3B, LK03.L125I and LK03 vector. Liver was harvested 2 weeks later for GFP expression analysis. Scale bar: 200 μm.

FIG. 2 shows differential transduction of human and mouse hepatocytes by AAV vectors. FRG mice were transduced with 3×10″ GC of AAV vectors expressing GFP. Livers were isolated from animals 21 days post vector administration, sectioned and stained for human fumaryl acetoacetate hydrolase (hFAH). Images were obtained using a NIKON inverted microscope using a 20× objective and equipped with a digital camera. A digital merge of the GFP and hFAH images is shown on the right panels.

FIG. 3 shows transduction efficiency (GFP) in NHP liver. Male rhesus macaques received 3×10¹² GC/kg of AAV3B, LK03.L125I, LK03, rh10, AAV8 or AAV2.TBG.GFP vector. Liver was harvested 10 (AAV3B, LK03.L125I, LK03, and AAV2) or 7 days (AAVrh10 and AAV8) post vector administration for GFP expression analysis (a). Scale bar: 200 μm. Transduction efficiency in NHP liver was evaluated by (b) morphometric analysis of the transduction efficiency based on percent transduction of hepatocytes, (c) morphometric analysis of the transduction efficiency based on relative GFP intensity, (d) quantification of GFP protein concentration in liver lysate by ELISA, and (e) vector genome copies in liver. *Note: data on AAV8 (previously published) are included for comparison purpose [Wang, L., et al, Hum Gene Ther, 22: 1389-1401 (2011)].

FIG. 4 shows biodistribution of AAV vector DNA in tissues of rhesus macaques following intravenous infusion of AAV3B, LK03.L125I, LK03, AAVrh10, AAV8, and AAV2. Tissues were harvested 10 (AAV3B, LK03.L125I, LK03, and AAV2) or 7 days (AAVrh10 and AAV8) post vector administration, total DNA prepared, and AAV DNA quantified by Taqman PCR. (a) The data are presented as vector DNA copies per microgram of total DNA. (b) Vector genome copies in liver and spleen are also presented as vector DNA copies per diploid genome.

FIG. 5 shows detection of AAV capsid within germinal centers of spleen by immunofluorescence (red) following systemic administration of AAV. Sections were counterstained with DAPI (blue) to outline splenic structure. Inset shows germinal center at higher magnification. Serotype-specific antisera were also used to stain spleen from a naïve animal or were omitted as control (RQ9175, lower panel). Scale bar: 400 μm.

FIG. 6 shows profiles of neutralizing antibodies. (a) Prevalence of neutralizing antibodies against AAV3B, AAVLK03.L125I, AAVLK03, AAVrh10, and AAV8 viruses was determined by an in vitro neutralization assay using AAV3B, AAVLK03.L125I, AAVLK03, AAVrh10, and AAV8.CMV.LacZ vectors. Sera from 28 normal human subjects from the US were tested for their ability to neutralize the transduction of each of the AAV viruses as described in the examples herein. (b) Cross reactivity of neutralizing antibodies of known AAVs (AAV1-9 and AAVrh10) to AAV3B, AAVLK03.L125I, AAVLK03, AAVrh10, and AAV8. Rabbits were immunized with intramuscular injections of 1×10¹³ GC of each of the AAV serotypes and boosted 34 days later with the same dose. Sera were analyzed for the presence of neutralizing antibodies by incubating serial 2-fold dilutions with 1×10⁹ GC of each appropriate AAV vector expressing LacZ. The serum dilution that produced a 50% reduction of LacZ expression was scored as the neutralizing antibody titer against that particular virus.

FIG. 7 provides a surface rendering of the VP3 subunit illustrating the differences between AAV8 and AAVrh10 (a), and AAV8 and AAV3B (b). In the left part of each panel, different colors indicate the differences in hypervariable regions I-IX relative to the AAV8 VP3 monomer (PDB: 2QA0) [Nam, H J, et al, J Virol 81: 12260-12271]. In the right part of each panel, the differences on the surface of the capsid are shown in red. The models are generated with Chimera program [Pettersen, E F, et al, (2004) J Comput Chem 25: 1605-1612; Sanner, M F, et al, (1996), Biopolymers 38: 305-320].

FIG. 8 provides in vitro transduction efficiency on Huh7 cells. Huh7 cells were co-infected with wild type adenovirus (MOI=45) and AAV3B, LK03.L125I, LK03, rh10 or AAV8.CMV.LacZ vectors at the MOI of 1,000 (solid bar) and 10,000 (hatched bar). Beta-galactosidase activity was assayed 24 hours after infection.

FIG. 9 provides gating strategy for evaluation of GFP expression in isolated hepatocytes. Hepatocytes were isolated using a dual perfusion collagenase protocol. Isolated hepatocytes were stained with antibodies against human HLA (a) or mouse H2-k^{b} (c). The subset of GFP transduced cells within the gated human (b) or mouse (d) class I positive cells was quantified using a flow cytometer.

FIG. 10 provides the vector genome distribution among the AVB column fractions. AAV vectors were diluted in binding buffer AVB.A (for AAV3B, culture supernatant was buffer-exchanged into the binding buffer) and then loaded onto the AVB column. Fractions from flow through (FT), AVB.A wash (W1), AVB.C wash (W2) and elution (AVB.B) (E) were collected. Vector genome copies were determined by real-time PCR.

FIGS. 11A-11B provide an AAV serotype sequence alignment. (a) The alignment was performed with Vector NTI using ClustalW algorithm. The 665-670 region (AAV8 vp1 numbering, SEQ ID NO:1) is shown with the SPAKFA epitope of AAV3B underlined. (b) The region corresponding to SPAKFA is shown in black on AAV8 capsid.

FIGS. 12A-12E illustrate the substitution mutant vector genome distribution among the AVB column fractions. AAV vectors and their SPAKFA mutants were loaded onto an AVB column. Fractions for flow through (FT), DPBS wash (W1), AVB.C wash (W2) and elution (E) were collected for real-time PCR titration and represented as percent genome copies of the total. Each AAV and its mutant were compared head-to-head from production to titration. For AAV8, AAV9 and rh.64R1 mutants were made by substituting the corresponding region to SPAKFA based on sequence alignments shown in FIG. 11A. For the AAV3B mutant, the SPAKFA epitope was replaced by NKDKLN [SEQ ID NO:2].

FIG. 13 provides the Huh7 cell transduction of AAVs and their SPAKFA mutants. The transgene cassette was CB7.CI.ffluciferase. Huh7 cells were infected with AAV vectors (filled circles) and their SPAKFA mutants (empty circles) at various concentrations (x-axis). The substitution mutant for AAV3B was AAV3B-NKDKLN [SEQ ID NO:2]. Luciferase expression was read 3 days after infection and denoted as RLU/s. RLU: Relative Luminescence Unit; gc: vector genome copies.

FIGS. 14A-14B provide the AAV capsid 328-333 region of vp1, based on the numbering of AAV8 [SEQ ID NO:1]. The sequence alignment of the 328-338 region of AAV8 VP1 is shown in (a) with the 328 and 333 residues of AAV8 [SEQ ID NO: 1] underlined. Included are the epitopes of AAV1 [SEQ ID NO: 17]; AAV2 [SEQ ID NO: 18]; AAV3B [with reference to SEQ ID NO: 3]; AAV5 [SEQ ID NO: 19]; AAVrh10 [SEQ ID NO: 20], AAVhu37 [SEQ ID NO: 21]; AAV8 [with reference to SEQ ID NO:1]; AAVrh64R1 [SEQ ID NO: 22]; and AAV9 [SEQ ID NO: 23]. Panel (b) demonstrates the two residues on AAV8 crystal structure. Two neighboring monomers of AAV8 capsid are shown (light and dark gray). The light, dashed pentagon indicates the pore. The dark gray region is the 665-670 region [with reference to the numbering of SEQ ID NO:1] of the light gray monomer.

### DETAILED DESCRIPTION OF THE INVENTION

Compositions and methods utilizing AAV vectors having AAV3-related capsids for liver directed therapies in human are provided. These AAV3-related capsids may be used as first-administration, e.g., where subsequent AAV related therapy is anticipated. This method is particularly useful where the regimen will utilize clade E based AAV vectors for targeting the liver in vivo and/or where the subject has pre-existing immunity to Clade E AAV. The compositions and methods described herein are also useful in treating patients which have neutralizing cross-reactivity to AAV from clades other than Clade E and which are not neutralizing for AAV3B based vectors.

In another aspect, the invention provides altered AAV capsids having at least one engineered purification (e.g., SPAKFA) epitope and methods for purifying AAV by engineering a purification epitope into an AAV capsid.

Vectors based on AAV3B yielded very interesting profiles of activities. The extremely low transduction achieved in mice in vivo with AAV3B vectors prevented their practical development since most of the disease models are in mice. The studies described herein indicated that AAV3B vectors are capable of very high in vivo transduction of human hepatocytes in the human liver xenograft model and macaque hepatocytes in macaque liver.

As used herein “transduction” refers to the process by which the expression cassette carrying the gene product is introduced into the target cells. “High in vivo transduction” refers to the levels of expression cassette delivered to hepatocytes (or splenic cells) via AAV3B vectors as described herein are higher than those achieved by other AAV vectors. Typically, transduction levels are measured by assessing gene product expressed in the target tissue or by measuring circulating transgene product in the case of a gene secreted from a transduced cell. A variety of methods are known for quantifying percentages of transduced cells (e.g., hepatocytes). Transduction can be evaluated by flow cytometric analysis of isolated hepatocytes (FACS) or by sectioning whole livers. For example, a method such as that described in the working example may be used in which images from each xenograft liver were taken for each channel (GFP and FAH stain). The percentage of image area positive for each protein was determined by thresholding with ImageJ software. Next, the thresholded images showing gene expression are combined with the corresponding thresholded images showing FAH-positive area or FAH-negative area. This was achieved with ImageJ's “Image Calculator” tool by image addition of thresholded images where the thresholded pixels equal 0 and the non-thresholded pixels equal 255 (so that 0+0=0, i.e. only the overlap area between two images remains the value 0 in the resulting image). The overlap area (i.e., pixels with value 0 showing positive cells) was then quantified and the percentage of expression-positive cells determined. In one embodiment, AAV3B-mediated delivery may result in at least about 10% to about 70% transduction levels in hepatocytes, or about 20% to about 60%, or about 25% to about 40%. Alternatively, in a patient, transduction levels can be assessed by measuring circulating levels of the product carried by the expression cassette.

As used herein, an AAV3-related capsid, “an AAV3B” vector or an “AAV3B” capsid, refers to AAV3B [U.S. Pat. No. 6,156,305 (amino acid sequence in SEQ ID ID:10 therein; crystal structure provided in Lerch, et al, 2010, Virology 403 (1), 26-36], and variants thereof, including AAV3B.ST [S663V+T492V modified AAV3B, reproduced herein as SEQ ID NO:6; Li Zhong et al, Abstract 240. American Society of Gene & Cell Therapy 17th Annual Meeting, 2014, Mol Therapy, Vol 22 (Suppl 1) May 2014, p. S91]; LK03[US 2013/0059732, see, SEQ ID NO: 31 for amino acid sequence, similar to AAV3B with only 8 amino differences between the two capsids, only one of which is located in the VP3 capsid, reproduced herein as SEQ ID NO:4], LK03 I125 [a variant of LK03 in which the Leu located at position 125 is substituted with an Ile (called AAVLK03.L125I), reproduced as SEQ ID NO:5].

The amino acid sequence of AAV3B, GenBank: AAB95452.1, citing Rutledge and Russell, J Virol, 72(1): 309-319 (1999), is reproduced in SEQ ID NO: 3: and provided below using single letter code:

>gi|2766610|gb|AAB95452.1|capsid protein VP1
[adeno-associated virus 3B]
MAADGYLPDWLEDNLSEGIREWWALKPGVPQPKANQQHQDNRRGLVLPGY
KYLGPGNGLDKGEPVNEADAAALEHDKAYDQQLKAGDNPYLKYNHADAEF
QERLQEDTSFGGNLGRAVFQAKKRILEPLGLVEEAAKTAPGKKRPVDQSP
QEPDSSSGVGKSGKQPARKRLNFGQTGDSESVPDPQPLGEPPAAPTSLGS
NTMASGGGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKKLSFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNRTQG
TTSGTTNQSRLLFSQAGPQSMSLQARNWLPGPCYRQQRLSKTANDNNNSN
FPWTAASKYHLNGRDSLVNPGPAMASHKDDEEKFFPMHGNLIFGKEGTTA
SNAELDNVMITDEEEIRTTNPVATEQYGTVANNLQSSNTAPTTRTVNDQG
ALPGMVWQDRDVYLQGPIWAKIPHTDGHFHPSPLMGGFGLKHPPPQIMIK
NTPVPANPPTTFSPAKFASFITQYSTGQVSVEIEWELQKENSKRWNPEIQ
YTSNYNKSVNVDFTVDTNGVYSEPRPIGTRYLTRNL. See, also,
http://www.ncbi.nlm.nih.gov/protein/-
2766610?report=genbank &log$=
protalign&blast_rank=1&RID = UE5YDH83015

This sequence represents the amino acid sequence of the vp1 protein, amino acids 1 to 736. The vp2 and vp3 proteins are splice variants thereof, wherein the vp2 protein is located about amino acids 138 to about 736 and the vp3 protein is located at about amino acids 203 to about 736 [see, SEQ ID NO: 3]. The vp1-unique region is refers to that portion of the capsid which are not present in vp2 or vp3, i.e., about amino acid 1 to about residue 137. The “vp2-unique region” refers to that portion of the capsid which is not present in vp3, i.e., about residue 138 to about 202.

The following sequence is that of AAV LK03, also reproduced in SEQ ID NO: 4:

Met Ala Ala Asp Gly Tyr Leu Pro Asp Trp Leu Glu
Asp Asn Leu Ser Glu Gly Ile Arg Glu Trp Trp Ala
Leu Gln Pro Gly Ala Pro Lys Pro Lys Ala Asn Gln
Gln His Gln Asp Asn Ala Arg Gly Leu Val Leu Pro
Gly Tyr Lys Tyr Leu Gly Pro Gly Asn Gly Leu Asp
Lys Gly Glu Pro Val Asn Ala Ala Asp Ala Ala Ala
Leu Glu His Asp Lys Ala Tyr Asp Gln Gln Leu Lys
Ala Gly Asp Asn Pro Tyr Leu Lys Tyr Asn His Ala
Asp Ala Glu Phe Gln Glu Arg Leu Lys Glu Asp Thr
Ser Phe Gly Gly Asn Leu Gly Arg Ala Val Phe Gln
Ala Lys Lys Arg Leu Leu Glu Pro Leu Gly Leu Val
Glu Glu Ala Ala Lys Thr Ala Pro Gly Lys Lys Arg
Pro Val Asp Gln Ser Pro Gln Glu Pro Asp Ser Ser
Ser Gly Val Gly Lys Ser Gly Lys Gln Pro Ala Arg
Lys Arg Leu Asn Phe Gly Gln Thr Gly Asp Ser Glu
Ser Val Pro Asp Pro Gln Pro Leu Gly Glu Pro Pro
Ala Ala Pro Thr Ser Leu Gly Ser Asn Thr Met Ala
Ser Gly Gly Gly Ala Pro Met Ala Asp Asn Asn Glu
Gly Ala Asp Gly Val Gly Asn Ser Ser Gly Asn Trp
His Cys Asp Ser Gln Trp Leu Gly Asp Arg Val Ile
Thr Thr Ser Thr Arg Thr Trp Ala Leu Pro Thr Tyr
Asn Asn His Leu Tyr Lys Gln Ile Ser Ser Gln Ser
Gly Ala Ser Asn Asp Asn His Tyr Phe Gly Tyr Ser
Thr Pro Trp Gly Tyr Phe Asp Phe Asn Arg Phe His
Cys His Phe Ser Pro Arg Asp Trp Gln Arg Leu Ile
Asn Asn Asn Trp Gly Phe Arg Pro Lys Lys Leu Ser
Phe Lys Leu Phe Asn Ile Gln Val Lys Glu Val Thr
Gln Asn Asp Gly Thr Thr Thr Ile Ala Asn Asn Leu
Thr Ser Thr Val Gln Val Phe Thr Asp Ser Glu Tyr
Gln Leu Pro Tyr Val Leu Gly Ser Ala His Gln Gly
Cys Leu Pro Pro Phe Pro Ala Asp Val Phe Met Val
Pro Gln Tyr Gly Tyr Leu Thr Leu Asn Asn Gly Ser
Gln Ala Val Gly Arg Ser Ser Phe Tyr Cys Leu Glu
Tyr Phe Pro Ser Gln Met Leu Arg Thr Gly Asn Asn
Phe Gln Phe Ser Tyr Thr Phe Glu Asp Val Pro Phe
His Ser Ser Tyr Ala His Ser Gln Ser Leu Asp Arg
Leu Met Asn Pro Leu Ile Asp Gln Tyr Leu Tyr Tyr
Leu Asn Arg Thr Gln Gly Thr Thr Ser Gly Thr Thr
Asn Gln Ser Arg Leu Leu Phe Ser Gln Ala Gly Pro
Gln Ser Met Ser Leu Gln Ala Arg Asn Trp Leu Pro
Gly Pro Cys Tyr Arg Gln Gln Arg Leu Ser Lys Thr
Ala Asn Asp Asn Asn Asn Ser Asn Phe Pro Trp Thr
Ala Ala Ser Lys Tyr His Leu Asn Gly Arg Asp Ser
Leu Val Asn Pro Gly Pro Ala Met Ala Ser His Lys
Asp Asp Glu Glu Lys Phe Phe Pro Met His Gly Asn
Leu Ile Phe Gly Lys Glu Gly Thr Thr Ala Ser Asn
Ala Glu Leu Asp Asn Val Met Ile Thr Asp Glu Glu
Glu Ile Arg Thr Thr Asn Pro Val Ala Thr Glu Gln
Tyr Gly Thr Val Ala Asn Asn Leu Gln Ser Ser Asn
Thr Ala Pro Thr Thr Arg Thr Val Asn Asp Gln Gly
Ala Leu Pro Gly Met Val Trp Gln Asp Arg Asp Val
Tyr Leu Gln Gly Pro Ile Trp Ala Lys Ile Pro His
Thr Asp Gly His Phe His Pro Ser Pro Leu Met Gly
Gly Phe Gly Leu Lys His Pro Pro Pro Gln Ile Met
Ile Lys Asn Thr Pro Val Pro Ala Asn Pro Pro Thr
Thr Phe Ser Pro Ala Lys Phe Ala Ser Phe Ile Thr
Gln Tyr Ser Thr Gly Gln Val Ser Val Glu Ile Glu
Trp Glu Leu Gln Lys Glu Asn Ser Lys Arg Trp Asn
Pro Glu Ile Gln Tyr Thr Ser Asn Tyr Asn Lys Ser
Val Asn Val Asp Phe Thr Val Asp Thr Asn Gly Val
Tyr Ser Glu Pro Arg Pro Ile Gly Thr Arg Tyr Leu
Thr Arg Pro Leu

Other variants may be generated which have at least 95% identity to the vp3 sequence of AAV3B at the amino acid level, more preferably at least 97% identity, or at least 99% identity. In one embodiment, these variations may include amino acid changes in the conserved regions of the capsid (e.g., contained primarily within the vp1- and/or the vp2-unique regions of the capsid amino acid sequence) which preserve the function of these regions which includes the ability to self-assemble and package an expression cassette, and no amino acid changes in the hypervariable regions of the capsid (e.g., no changes in the vp3-unique region of the capsid). In another embodiment, there are amino acid changes in the vp3-unique fewer than 10 amino acid changes, fewer than 7 amino acid changes, or only 1, 2, 3, 4 or 5 amino acid changes. In another embodiment, these AAV3B capsids may modified, e.g., as described in WO 2008/027084, to ablate the heparin binding site. Vectors based on these AAV may be produced using some or all of the methods described in US 2009/0275107.

As used herein, a clade E AAV is as defined in US 2011/0236353, which is hereby incorporated by reference. This clade is characterized by containing the previously described AAV8 [G. Gao et al, Proc. Natl Acad. Sci USA, 99:11854-9 (Sep. 3, 2002)], 43.1/AAVrh2; 44.2/AAVrh10; AAVrh25; 29.3/AAVbb.1; and 29.5/AAVbb.2 [US Published Patent Application No. US 2003/0138772 A1 (Jul. 24, 2003)]. Further, the clade novel AAV sequences, including, without limitation, including, e.g., 30.10/AAVpi.1, 30.12/pi.2, 30.19/pi.3, LG-4/rh.38; LG-10/rh.40; N721-8/rh.43; 1-8/rh.49; 2-4/rh.50; 2-5/rh.51; 3-9/rh.52; 3-11/rh.53; 5-3/rh.57; 5-22/rh.58; 2-3/rh.61; 4-8/rh.64; 3.1/hu.6; 33.12/hu.17; 106.1/AAVhu37; LG-9/hu.39; 114.3/hu. 40; 127.2/hu.41; 127.5/hu.42; AAVhu66; and AAVhu67. This clade further includes modified AAVrh2; modified AAVrh58; modified AAVrh64. A clade is a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described extensively in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in clade E identified herein or is outside this. While clade E as defined herein is based primarily upon naturally occurring AAV vp1 capsids, the clades are not limited to naturally occurring AAV. The clades can encompass non-naturally occurring AAV, including, without limitation, recombinant, modified or altered, chimeric, hybrid, synthetic, artificial, etc., AAV which are phylogenetically related as determined using a Neighbor-Joining algorithm at least 75% (of at least 1000 replicates) and a Poisson correction distance measurement of no more than 0.05, based on alignment of the AAV vp1 amino acid sequence.

Generally, when referring to “identity” between two different adeno-associated viruses, “identity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments may be performed using any of a variety of publicly or commercially available Multiple Sequence Alignment Programs. Examples of such programs include, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

The term “serotype” is a distinction with respect to an AAV having a capsid which is serologically distinct from other AAV serotypes. Serologic distinctiveness is determined on the basis of the lack of cross-reactivity between antibodies to the AAV as compared to other AAV. Cross-reactivity is typically measured in a neutralizing antibody assay. A “neutralizing antibody” or “Nab” is an antibody which prevents an antigen or infectious body by inhibiting or “neutralizing” its biological effect. In one embodiment, a neutralizing antibody assay uses polyclonal serum generated against a specific AAV in a rabbit or other suitable animal model using the adeno-associated viruses. In this assay, the serum generated against a specific AAV is then tested in its ability to neutralize either the same (homologous) or a heterologous AAV. The dilution that achieves 50% neutralization is considered the neutralizing antibody titer. If for two AAVs the quotient of the heterologous titer divided by the homologous titer is lower than 16 in a reciprocal manner, those two vectors are considered as the same serotype. Conversely, if the ratio of the heterologous titer over the homologous titer is 16 or more in a reciprocal manner the two AAVs are considered distinct serotypes.

The term “proliferating cells” as used herein refers to cells which multiply or reproduce, as a result of cell growth and cell division. Cells may be naturally proliferating at a desired rate, e.g., epithelial cells, stem cells, blood cells, hepatocytes.

In general, a neonate in humans may refer to infants from birth to under about 28 days of age; and infants may include neonates and span up to about 1 year of age to up to 2 years of age. The term “young children” may span to up to about 11-12 years of age.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% (±10%) from the reference given, unless otherwise specified.

A “subject” is a mammal, e.g., a human, mouse, rat, guinea pig, dog, cat, horse, cow, pig, or non-human primate, such as a monkey, chimpanzee, baboon or gorilla. A patient refers to a human. A veterinary subject refers to a non-human mammal.

As used herein, “disease”, “disorder” and “condition” are used interchangeably, to indicate an abnormal state in a subject.

A recombinant AAV vector (AAV viral particle) comprises, packaged within an AAV capsid, a nucleic acid molecule containing a 5′ AAV ITR, the expression cassettes described herein and a 3′ AAV ITR. As described herein, an expression cassette contains one or more an open reading frame(s) operably linked to regulatory elements which direct expression thereof in a transduced host cell (e.g., a hepatocyte). One or more of the elements of the expression cassette are exogenous to the AAV capsid.

Use of rAAV vectors having AAV3B capsids used alone or in regimens with Clade E based vectors are described herein, as AAV which preferentially target the liver and/or deliver genes with high efficiency are particularly desired. However, the regimens or methods may utilize other vectors having different AAV capsids. Further, the rAAV vectors described herein, having mutant binding epitopes to facilitate purification, may be used as a sole active component, or in a regimen with other rAAV or other active components, for a variety of gene delivery therapies or vaccines, for targeting liver or other suitable cells.

The sequences of the AAV3B capsids are as defined above. Further, the sequences of Clade E vectors such as AAV8 and rh10 have been described in, e.g., U.S. Pat. No. 7,790,449; U.S. Pat. No. 7,282,199, WO 2003/042397, and a variety of databases. Still other AAV sources may include, e.g., AAV9 [U.S. Pat. No. 7,906,111; US 2011-0236353-A1], and/or hu37 [see, e.g., U.S. Pat. No. 7,906,111; US 2011-0236353-A1], AAV1, AAV2, AAV3, AAV4, AAV5, AAV6, AAV6.2, AAV7, AAV8, [U.S. Pat. No. 7,790,449; U.S. Pat. No. 7,282,199] and others. See, e.g., WO 2003/042397; WO 2005/033321, WO 2006/110689; U.S. Pat. No. 7,790,449; U.S. Pat. No. 7,282,199; U.S. Pat. No. 7,588,772B2 for sequences of these and other suitable AAV, as well as for methods for generating AAV vectors. Still other AAV may be selected, optionally taking into consideration tissue preferences of the selected AAV capsid.

The AAV vector may contain a full-length AAV 5′ inverted terminal repeat (ITR) and a full-length 3′ ITR. A shortened version of the 5′ ITR, termed ΔITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Where a pseudotyped AAV is to be produced, the ITRs are selected from a source which differs from the AAV source of the capsid. For example, AAV2 ITRs may be selected for use with an AAV capsid having a particular efficiency for a selected cellular receptor, target tissue or viral target. In one embodiment, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

A single-stranded AAV viral vector may be used. Methods for generating and isolating AAV viral vectors suitable for delivery to a subject are known in the art. See, e.g., U.S. Pat. No. 7,790,449; U.S. Pat. No. 7,282,199; WO 2003/042397; WO 2005/033321, WO 2006/110689; and U.S. Pat. No. 7,588,772 B2]. In one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following US patents, the contents of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The AAV may be prepared as described in, e.g., US Published Patent Application No. 2009/0275107, which provides an optionally continuous process for producing AAV and isolating from cell culture without requiring cell permeabilization and/or cell lysis. Alternatively, AAV3B-based rAAV vectors or rAAV with engineered capsids as described herein may be purified using the methods described herein.

In one embodiment, the rAAV described herein are designed for expressing its gene product in hepatocytes. In addition to the AAV 5′ ITR and 3′ ITR, the open reading frame(s) of the expression cassette may include tissue-specific regulatory elements, regulatable elements, or constitutive elements.

The expression cassette typically contains a promoter sequence as part of the expression control sequences, e.g., located between the selected 5′ ITR sequence and the coding sequence. In one embodiment, the promoter may be the liver-specific promoter thyroxin binding globulin (TBG). Alternatively, other liver-specific promoters may be used [see, e.g., The Liver Specific Gene Promoter Database, Cold Spring Harbor, http://rulai.cshl.edu/LSPD/, such as, e.g., alpha 1 anti-trypsin (A1AT); human albumin Miyatake et al., J. Virol., 71:5124 32 (1997), humA1b; and hepatitis B virus core promoter, Sandig et al., Gene Ther., 3:1002 9 (1996)]. TTR minimal enhancer/promoter, alpha-antitrypsin promoter, LSP (845 nt)25 (requires intron-less scAAV); or LSP1. Other promoters, such as constitutive promoters, regulatable promoters [see, e.g., WO 2011/126808 and WO 2013/04943], or a promoter responsive to physiologic cues may be used may be utilized in the vectors described herein.

In addition to a promoter, an expression cassette and/or a vector may contain one or more other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, e.g., SV40, SV50, bovine growth hormone (bGH), human growth hormone, and synthetic polyAs. Examples of suitable enhancers include, e.g., the alpha fetoprotein enhancer, the TTR minimal promoter/enhancer, LSP (TH-binding globulin promoter/alpha1-microglobulin/bikunin enhancer), amongst others. In one embodiment, the expression cassette comprises one or more expression enhancers. In one embodiment, the expression cassette contains two or more expression enhancers. These enhancers may be the same or may differ from one another. For example, an enhancer may include an Alpha mic/bik enhancer. This enhancer may be present in two copies which are located adjacent to one another. Alternatively, the dual copies of the enhancer may be separated by one or more sequences. In still another embodiment, the expression cassette further contains an intron, e.g., the Promega intron. Other suitable introns include those known in the art, e.g., such as are described in WO 2011/126808. Optionally, one or more sequences may be selected to stabilize mRNA. An example of such a sequence is a modified WPRE sequence, which may be engineered upstream of the polyA sequence and downstream of the coding sequence [see, e.g., M A Zanta-Boussif, et al, Gene Therapy (2009) 16: 605-619.

These control sequences are “operably linked” to the coding sequences. As used herein, the term “operably linked” refers to both expression control sequences that are contiguous with the gene of interest and expression control sequences that act in trans or at a distance to control the gene of interest.

A variety of different diseases and conditions may be treated using the method described herein. Examples of such conditions may include, e.g., alpha-1-antitrypsin deficiency, liver conditions (e.g., biliary atresia, Alagille syndrome, alpha-1 antitrypsin, tyrosinemia, neonatal hepatitis, Wilson disease), metabolic conditions such as biotinidase deficiency, carbohydrate deficient glycoprotein syndrome (CDGS), Crigler-Najjar syndrome, diabetes insipidus, Fabry, galactosemia, glucose-6-phosphate dehydrogenase (G6PD), fatty acid oxidation disorders, glutaric aciduria, hypophosphatemia, Krabbe, lactic acidosis, lysosomal storage diseases, mannosidosis, maple syrup urine, mitochondrial, neuro-metabolic, organic acidemias, PKU, purine, pyruvate dehydrogenase deficiency, urea cycle conditions, vitamin D deficient, and hyperoxaluria. Urea cycle disorders include, e.g., N-acetylglutamate synthase deficiency, carbamoyl phosphate synthetase I deficiency, ornithine transcarbamylase deficiency, “AS deficiency” or citrullinemia, “AL deficiency” or argininosuccinic aciduria, and “arginase deficiency” or argininemia.

Other diseases may also be selected for treatment according to the method described herein. Such diseases include, e.g., cystic fibrosis (CF), hemophilia A (associated with defective factor VIII), hemophilia B (associated with defective factor IX), mucopolysaccharidosis (MPS) (e.g., Hunter syndrome, Hurler syndrome, Maroteaux-Lamy syndrome, Sanfilippo syndrome, Scheie syndrome, Morquio syndrome, other, MPSI, MPSII, MPSIII, MSIV, MPS 7); ataxia (e.g., Friedreich ataxia, spinocerebellar ataxias, ataxia telangiectasia, essential tremor, spastic paraplegia); Charcot-Marie-Tooth (e.g., peroneal muscular atrophy, hereditary motor sensory neuropathy), glycogen storage diseases (e.g., type I, glucose-6-phosphatase deficiency, Von Gierke), II (alpha glucosidase deficiency, Pompe), III (debrancher enzyme deficiency, Cori), IV (brancher enzyme deficiency, Anderson), V (muscle glycogen phosphorylase deficiency, McArdle), VII (muscle phosphofructokinase deficiency, Tauri), VI (liver phosphorylase deficiency, Hers), IX (liver glycogen phosphorylase kinase deficiency). This list is not exhaustive and other genetic conditions are identified, e.g., www.kumc.edu/gec/support; www.genome.gov/10001200; and www.ncbi.nlm.nih.gov/books/NBK22183/, which are incorporated herein by reference.

The compositions described herein are designed for delivery to subjects (e.g., human patients) in need thereof by any suitable route or a combination of different routes. For treatment of liver disease, direct or intrahepatic delivery to the liver is desired and may optionally be performed via intravascular delivery, e.g., via the portal vein, hepatic vein, bile duct, or by transplant. Alternatively, other routes of administration may be selected (e.g., oral, inhalation, intranasal, intratracheal, intraarterial, intraocular, intravenous, intramuscular, and other parental routes). For example, intravenous delivery may be selected for delivery to proliferating, progenitor and/or stem cells. Alternatively, another route of delivery may be selected. Optionally, the rAAV vectors described herein may be delivered in conjunction with other viral vectors, or non-viral DNA or RNA transfer moieties. The vectors (or other transfer moieties) can be formulated with a physiologically acceptable carrier for use in gene transfer and gene therapy applications. In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually poly A signal). The rAAV virus can be formulated in dosage units to contain an amount of rAAV that is in the range of about 1.0×10⁹ GC to about 1.0×10¹⁵ GC (to treat an average subject of 70 kg in body weight), and preferably 1.0×10¹² GC to 1.0×10¹⁴ GC for a human patient. Preferably, the dose of replication-defective virus in the formulation is 1.0×10⁹ GC, 5.0×10⁹ GC, 1.0×10¹⁰ GC, 5.0×10¹⁰ GC, 1.0×10¹¹ GC, 5.0×10¹¹ GC, 1.0×10¹² GC, 5.0×10¹² GC, or 1.0×10¹³ GC, 5.0×10¹³ GC, 1.0×10¹⁴ GC, 5.0×10¹⁴ GC, or 1.0×10¹⁵ GC.

The above-described recombinant vectors or other constructs may be delivered to host cells according to published methods. The vectors or other moieties are preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In one embodiment, the rAAV3B compositions may be used in regimens, including the methods described in the Crispr/Cas methods described in PCT/US16/29330, filed Apr. 26, 2016, US Provisional Patent Application Nos. 62/287,511, filed Jan. 27, 2016, U.S. Provisional Patent Application No. 62/254,225, filed Nov. 12, 2015, U.S. Provisional Patent Application No. 62/183,825, filed Jun. 24, 2015, and US Provisional Patent Application No. 62/153,470, filed Apr. 27, 2015, which are incorporated herein by reference. In another embodiment, the rAAV3B compositions are used in regimens for treating ornithine transcarbamylase (OTC) deficiency, treating fibrosis or cirrhosis in a subject heterozygous for OTC deficiency, and/or preventing and/or treating hepatocellular carcinoma in a subject heterozygous for ornithine transcarbamylase deficiency, e.g., as described in PCT/US15/19536, filed Mar. 9, 2015, which is incorporated by reference herein.

In another embodiment, a regimen for delivery of a gene product to a human patient is provided. The regimen involves delivery of a first recombinant AAV vector comprising an expression cassette comprising an exogenous sequence encoding a gene product under control of regulatory sequences which direct expression thereof in a cell; and delivery of a second recombinant AAV vector comprising an expression cassette comprising an exogenous sequence encoding a gene product under control of regulatory sequences which direct expression of the product in a cell, wherein the first recombinant AAV vector or the second AAV vector has an AAV3B capsid. The rAAV vector(s) may be administered to the patient by any suitable route of delivery as described herein. In one embodiment, the first or second rAAV has a Clade E capsid, e.g., AAV8 or rh10. This regimen is particularly well suited to target liver cells in the patient. In one embodiment, the rAAV3B vector provides high transduction levels of hepatocytes post-administration, as compared to rAAV from other clades.

In one embodiment, the first AAV is delivered to neonatal patients. In this instance, a further dose may be delivered following the neonatal stage. This may be desired in order to address the dilution effect from rapidly proliferating hepatocytes which is present during the infancy and young childhood. Optionally, the delivery of the first rAAV and the second rAAV are temporally separated by at least one month, at least three months, or by at least about 1 year to about 10 years.

Optionally, a regimen such as described herein, includes delivery of at least a third AAV, wherein one of the administered rAAVs has an AAV3B capsid. Optionally, the AAV3B capsid is selected from AAV3B.

In a further embodiment, a method of providing high hepatocyte transduction levels is provided, which involves administering a rAAV3B based vector to the patient. Advantageously, this method is useful for patients having pre-existing immunity to AAV from Clade E or another rAAV type which preferentially targets the liver. Such pre-existing immunity may be the result of a natural exposure or previously administered rAAV.

In another aspect, the invention provides recombinant vectors having capsids with an engineered epitope useful for purifying the viral vector. More particularly, a virus vector having a capsid or envelope protein is engineered to contain a SPAKFA epitope which is not present in the virus capsid or envelope prior to being engineered to contain same. Also provides are methods for purifying the vector by engineering such an epitope into the viral capsid or envelope. The method is particularly well suited for rAAV. In one embodiment, the rAAV has a capsid which is engineered to contain a SPAKFA epitope. In one embodiment, the epitope is engineered into the vp3 capsid protein. For example, the epitope may be engineered into the region of the selected AAV (e.g., AAV1) which corresponds to the region of AAV3B which natively contains this epitope. For example, the epitope may be inserted in the residues of the selected AAV capsid which aligns with residues 665 to 670 based on the numbering of the AAV8 vp1 capsid (SEQ ID NO:1) and/or residues 664 to 668 of AAV3B (SEQ ID NO:3). In another embodiment, the epitope may be inserted in another location, e.g., fused to the carboxy- or amino-terminus of the vp3 capsid protein. In one embodiment, the vp2 protein is optionally present. In a further embodiment, the vp2 capsid protein is present and the epitope is fused to the carboxy- or amino-terminus of the vp2 capsid protein. In still another embodiment, the epitope is engineered into another location in the capsid.

In one embodiment, a rAAV vector having an engineered SPAKFA peptide in its capsid is provided. In such a rAAV, a capsid protein which lacks such an epitope is modified in one or more amino acid residues to have a SPAKFA epitope in the capsid region corresponding to amino acid residues 665 to 670, based on the numbering of the AAV8 vp1 capsid [SEQ ID NO:1] (residues 664-668 of AAV3B, SEQ ID NO:3). Additionally, an AAV capsid may further be provided with a threonine at position 333 (based on the numbering of AAV8, SEQ ID NO: 1). In addition to one or both of these modifications, a further engineered AAV capsid is heterologous to AAV3B, but engineered to contain the sequence of about amino acid residues 328 to about amino acid 333 of AAV3B [SEQ ID NO: 3]. Such an engineered capsid may contain this epitope as an alternative or in addition to the SPAKFA mutation and/or as an alternative or in addition to the threonine.

Methods of aligning AAV in order to determine the proper amino acid region in the AAV capsid targeted for modification have been described in the literature and/or are available through commercial vendors and web-based applications. See, e.g., discussion of multiple sequence alignment programs provided above in this document. As described herein, the numbering of AAV8 [see, e.g., Gao et al, PNAS USA, 99(18): 11854-11859 (2002); GenBank: AAN03857.1] is used as the reference point. However, another AAV may be selected as the reference, adjusting the residue numbers as appropriate based on the alignment of the selected reference AAV to AAV8.

Methods of altering the AAV may involve a variety of techniques, which techniques are known to those of skill in the art. For example, site directed mutagenesis may be performed at the level of the nucleic acids encoding one or more amino acids to be altered. Alternatively, an insertion of one or more amino acids (e.g., 2, 3, 4, 5 or more) may be made at the target region within the AAV capsid. Still other suitable techniques may be selected. See, e.g., Green and Sambrook, “Molecular Cloning: A Laboratory Manual, Cold Spring Harbor Press; 4^{th} Edition (Jun. 15, 2012). AAVs may be selected from among a variety of known AAV such as, e.g., those described in Still other AAV sources may include, e.g., AAV9 [U.S. Pat. No. 7,906,111; US 2011-0236353-A1], and/or hu37 [see, e.g., U.S. Pat. No. 7,906,111; US 2011-0236353-A1], AAV1, AAV2, AAV3, AAV4, AAV5, AAV6, AAV6.2, AAV7, AAV8, [U.S. Pat. No. 7,790,449; U.S. Pat. No. 7,282,199] and others. See, e.g., WO 2003/042397; WO 2005/033321, WO 2006/110689; U.S. Pat. No. 7,790,449; U.S. Pat. No. 7,282,199; U.S. Pat. No. 7,588,772B2 for sequences of these and other suitable AAV.

With these modifications in place, the engineered rAAV may be generated using methods described herein, or other methods described in the art, and purified as described. See, e.g., M. Mietzsch et al, “OneBac: Platform for Scalable and High-Titer Production of Adeno-Associated Virus Serotype 1-12 Vectors for Gene Therapy, Hum Gene Ther. 2014 Mar. 1; 25(3): 212-222. See, also, Smith R H, et al, Mol Ther, 2009 November; 17(11): 1888-96 (2009), describing a simplified baculovirus-AAV vector expression system coupled with one-step affinity purification. For example, lystates or supernatants (e.g., treated, freeze-thaw supernatants or media containing secreted rAAV), may be purified using one-step AVB sepharose affinity chromatography using 1 ml prepacked HiTrap columns on an ACTA purifier (GE Healthcare) as described by manufacturer, or in M. Mietzsch, et al, cited above.

For example, in one embodiment, an affinity capture method as provided herein is performed using an antibody-capture affinity resin. In one embodiment, the solid support is a cross-linked 6% agarose matrix having an average particle size of about 34 μm and having an AAV-specific antibody. An example of one such commercially available affinity resin is AVB Sepharose™ high performance affinity resin using an AAV-specific camelid-derived single chain antibody fragment of llama origin which is commercially available from GE Healthcare (AVB Sepharose). The manufacturer's literature further recommends up to a 150 cm/h flow rate and a relatively low loading salt concentration. Other suitable affinity resins may be selected or designed which contain an AAV-specific antibody, AAV1 specific antibody, or other immunoglobulin construct which is an AAV-specific ligand. Such solid supports may be any suitable polymeric matrix material, e.g., agarose, sepharose, sephadex, amongst others. Suitable loading amounts may be in the range of about 2 to about 5×10¹⁵ GC, or less, based on the capacity of a 30-mL column. Equivalent amounts may be calculated for other sized columns or other vessels.

Alternatively, the constructs used herein may be purified using other techniques known in the art.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not a limitation on the invention described herein.

### EXAMPLES

In an attempt to broaden the repertoire of capsids for liver gene therapy a thorough evaluation of vectors was conducted based on two previously described endogenous capsids, AAVrh10 and AAV3B, as well as the recently described engineered capsid AAVLK03, all of which were benchmarked against AAV2 and AAV8.

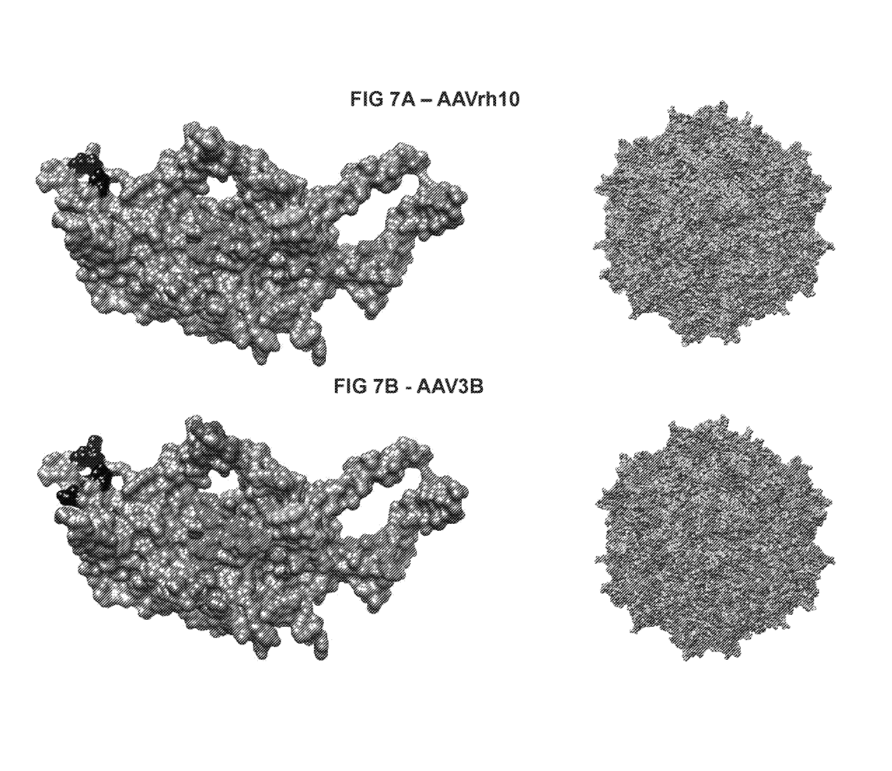

AAVrh10 was selected for this study because it is emerging as a lead capsid for clinical applications outside of the liver [NCT01161576. Safety Study of a Gene Transfer Vector (AAVrh10) for Children With Late Infantile Neuronal Ceroid Lipofuscinosis. ClinicalTrials.gov.; NCT01414985. AAVRh10 Administered to Children With Late Infantile Neuronal Ceroid Lipofuscinosis With Uncommon Genotypes or Moderate/Severe Impairment. Clinicaltrials.gov]. The expectation is that vectors based on AAVrh10 will have similar properties to AAV8 vectors since they are from the same clade and differ by only 8% in terms of the amino acid sequence of VP3. As shown in FIG. 7, these differences are localized primarily to the surface exposed hypervariable regions. AAV3B is quite distinct structurally and serologically from AAV8 (see FIG. 7 for summary of structural differences). Interestingly, capsids similar to AAV3B have rarely been recovered from natural sources, with the exception of one named as AAV (VR-942) which was isolated by PCR as a contaminant of simian adenovirus 17 [Schmidt, M., et al, J Virol, 82: 8911-8916 (2008)]. The closest family to AAV3B is clade C which is a collection of viruses formed from an AAV2/AAV3 hybrid. Not much work has been conducted with vectors based on AAV3B because of very low in vivo transduction efficiencies in murine models.

### Example 1

The results of the following studies are provided in FIGS. 7-9.
A. In vitro transduction. Human hepatoma cell line Huh7 was maintained in Dulbecco's modification of Eagle's medium (DMEM [Cellgro]) supplemented with 10% fetal bovine serum (FBS [Hyclone]). Cells were cultured at 37° C. with 5% CO₂ in the air and seeded to 96-well plates at the density of 1×10⁵ cells/well the day before in vitro transduction. Two hours before AAV transduction, cells were infected with wild-type adenovirus 5 (45 particles/cell). Cells were then transduced with AAV.CMV.LacZ vectors at the MOIs of 1,000 and 10,000, respectively (6 wells for each vector at each MOI). In vitro transduction efficiencies were evaluated 24 hours later by measuring β-galactosidase in cell lysate using mammalian β-galactosidase assay kit for bioluminescence, in accordance with the manufacturers' protocol (Applied Biosystems), and measured in a microplate luminometer (Clarity [BioTek]).
B. Flow cytometry. For flow cytometry analysis, 1 million hepatocytes were stained with PE-Cy7 conjugated anti-human HLA-A,B,C (BD Biosciences, San Jose, Calif.) and Alexa 647 conjugated anti-mouse H2-k^{b} (BD biosciences). Stained cells were washed and evaluated for percent transduced human or mouse hepatocytes by gating on the GFP⁺HLA⁺ or GFP⁺H2-K^{b+} cells, respectively. Samples were run on a Beckman Coulter flow cytometer (FC500) and the data analyzed using FlowJo.

### Example 2

A. Vectors

AAV vectors (AAV2, AAV3B, AAVLK03, AAVLK03.L125I, and AAVrh10 carrying the TBG.GFP.bGH or CMV.LacZ.bGH cassettes) were produced by the Vector Core at the University of Pennsylvania as previously described [Lock, M., et al, Hum Gene Ther, 21: 1259-1271 (2010)]. Vectors for macaque studies were subjected to extensive quality control tests including three repeated vector genome titrations based on qPCR, sodium dodecyl sulfate polyacrylamide gel electrophoresis (SDS—PAGE) analysis for vector purity, Limulus amebocyte lysate (LAL) for endotoxin detection (Cambrex Bio Science, East Rutherford, N.J.), and transgene expression analysis in mice and monkeys.

B. Murine Experiments.

All mice were housed in an AAALAC (Association for Assessment and Accreditation of Laboratory Animal Care)-accredited and PHS (Public Health Service)-assured facility at the University of Pennsylvania, and all animal procedures were performed in accordance with protocols approved by the Institute of Animal Care and Use Committees (IACUC) at the University of Pennsylvania. C57BL/6 male mice (6-8 weeks old) were purchased from Jackson Laboratories (Bar Harbor, Me.) and received a single tail vein injection of 1×10¹¹ or 3×10¹¹ genome copies of vector. GFP expressions were evaluated 2 weeks post vector injection. Male FRG mice on a C57BL/6N background and repopulated with 40-70% human hepatocytes, were purchased from Taconic-Yecuris (Tualatin, OR). Mice were provided ad libitum access to irradiated Purina Lab Diet 5115 (Ralston Purina Co., St. Louis, Mo.). According to the vendor's recommendations, all animals were initially maintained on a sterile solution of Nitisinone (2-(2-nitro-4-trifluoro-methylbenzoyl)1,3-cyclohexedione or NTBC, 8 mg/L, Yecuris, Tualatin, OR) and supplemented with Sulfamethoxazole (SMX, 640 mg/L; Yecuris) plus Trimethoprin (TMP, 128 mg/L, Yecuris) in 3% dextrose drinking water. Two weeks before vector administration, NTBC was withdrawn and animals were maintained on 3% dextrose drinking water supplemented with SMX/TMP. AAV.TBG.GFP vector (3×10″ GC) was intravenously administered and animals were put back on NTBC one week later. During NTBC withdrawal, mice that became dehydrated and/or lost ≥10% of their pre-shipment body weight were treated with fluid intervention and high-calorific diet (STAT, PRN Pharmacal, Pensacola, Fla.). Livers and hepatocytes were isolated three weeks post vector infusion.

C. Macaque Experiments.

Juvenile rhesus macaques (male Chinese origin and captive bred) were treated and cared for at an AAALAC-accredited and PHS-assured facility at the University of Pennsylvania (Philadelphia, Pa.) during the study. The study was performed according to a protocol approved by the Environmental Health and Radiation Safety Office, the Institutional Biosafety Committee, and the IACUC of the University of Pennsylvania. Vectors (3×10¹² GC/kg) were administered to the study animals via the saphenous vein in a total volume of 10 ml infused at 1 ml per minute using a Harvard® infusion pump. Blood samples were taken pre-study and at the time of necropsy via venipuncture of the femoral vein. At the time of necropsy, the target organ liver and 15 distant tissues (cerebrum, spinal cord, heart, lung, gallbladder, pancreas, spleen, kidney, testicles, stomach, duodenum, colon, mesenteric lymph nodes, bone marrow, and skeletal muscle-quadriceps femoris) were collected for vector biodistribution analysis.

D. Histology

To visualize GFP fluorescence, liver tissues were fixed overnight in formalin, washed in PBS, and frozen in OCT compound to produce cryosections (8 μm). GFP-positive liver area was quantified on representative images of cryosections from each animal (10× objective; 10 images for each NHP and a minimum of 3 images for each group of mice) using ImageJ software (W. Rasband, National Institutes of Health, Bethesda, Md.; http://rsb.info.nih.gov/ij). Images were set to a threshold to select GFP-positive hepatocytes and the percentage of GFP-positive liver area was then determined and averaged for each NHP or group of mice.

Liver sections from NHPs were further analyzed by measuring the fluorescence intensity of GFP. GFP intensity was measured as the total intensity of every image (i.e., the sum of all pixel values per image) determined with ImageJ software. The resulting intensity values were then calculated as a fraction of a fluorescence standard [Model, M A and Burkhard, J L, Cytometry, 44: 309-316 (2001), i.e., a 10% (w/v) solution of sodium fluorescein (Sigma-Aldrich, St. Louis, Mo.) in 0.1 M NaHCO₃. Images were taken from the reference solution spread on a slide and the original GFP intensity values were then divided by the reference values to obtain the final GFP intensity value. For each liver, 10 images were analyzed and mean values are presented.

Immunostaining on spleen sections was performed as described using rabbit sera made in our lab against the described serotypes [Wang, L., et al, Hum Gene Ther, 22: 1389-1401 (2011)]. AAVLK03 and LK03.L125I were detected using a rabbit serum raised against AAV3B.

Immunostaining on livers of xenograft mice was performed on formalin-fixed paraffin-embedded liver tissues. Paraffin sections were dewaxed and antigen retrieval was performed in citrate buffer pH6.0. Incubation with primary antibodies was performed after blocking with 1% donkey serum+0.2% Triton using chicken antibodies against GFP (Abcam, Cambridge, Mass.) and goat antibodies against FAH (Santa Cruz, Dallas, Tex.). After washing in PBS, the sections were stained with fluorescent-labeled secondary antibodies (Jackson Immunoresearch Laboratories, West Grove, Pa.) in 1% donkey serum for 30 min, washed again, and mounted with Vectashield plus DAPI (Vector Labs, Burlingame, Calif.).

To quantify percentages of transduced human and mouse hepatocytes, 10 images (10× objective) from each xenograft liver were taken for each channel (GFP and FAH stain). The percentage of image area positive for each protein was determined by thresholding with ImageJ software. Next, the thresholded images showing GFP expression were combined with the corresponding thresholded images showing human hepatocytes (i.e., FAH-positive area) or mouse hepatocytes (i.e., FAH-negative area not including cell-free areas such as veins). This was achieved with ImageJ's “Image Calculator” tool by image addition of thresholded images where the thresholded pixels equal 0 and the non-thresholded pixels equal 255 (so that 0+0=0, i.e. only the overlap area between two images remains the value 0 in the resulting image). The overlap area (i.e. pixels with value 0 showing GFP positive human or mouse cells) was then quantified and the percentage of GFP-positive human and mouse cells determined.

E. Hepatocyte Isolation

Mouse hepatocytes were isolated in a BSL-2 hood based on the in situ two-step collagenase perfusion technique [Model, M A and Burkhard, J K, Cytometry, 44: 309-316 (2001); Li, W C, et al, Methods Mol Biol, 633: 185-196 (2010)]. Briefly, the animal was anesthetized and opened up to expose the lower abdomen. The inferior vena cava was perfused for 5 min (retrograde perfusion) with Liver perfusion medium (Life Technologies, Grand Island, N.Y.). Once the perfusion was started the portal vein was cut to allow outflow of the perfusion. After 5 min the buffer was changed to collagenase medium containing 0.8 mg/mL Collagenase Type I (Worthington, Biochemical Corp., Lakewood, N.J.) in Hanks Balanced salt solution and perfused for an additional 12 minutes. The collagenase and perfusion buffers were maintained in a water bath set at 39° C. At the end of the perfusion the liver was excised and placed in Hepatocyte wash medium (Invitrogen) and the hepatocytes gently dispersed by teasing the tissue. The hepatocyte preparation was filtered through a 100 micron filter and washed three times and resuspended in hepatocyte wash medium.

F. Quantification of GFP Protein in Liver Lysate

GFP protein concentration in macaque liver lysate was measure by ELISA as previously described [Wang, L, et al, Hum Gene Ther, 22: 1389-1401 (2011)].

G. Vector Bio-Distribution Analysis

Tissue DNAs were extracted using QIAamp DNA Mini Kit (Qiagen, Valencia, Calif.). Detection and quantification of vector genomes in extracted DNA were performed by real-time PCR as described previously.

H. AAV Neutralizing Antibody Assay

Serum samples were collected and AAV NAb assays were performed on Huh7 cells as previously described [Calcedo, R., et al, J Infect Dis, 199: 381-390]. The limit of detection for the assay is 1:5 serum dilution.

I. Results/Discussion

Vectors expressing LacZ were evaluated for transduction of the human hepatoma cell line Huh7 at MOIs of 1,000 and 10,000 (FIG. 8). As has been previously noted, in vitro transduction with AAV3B is much higher than with AAV8; transduction with AAVrh10 was indistinguishable from that of AAV8. AAVLK03 and AAVLK03.L125I vectors showed transduction efficiencies equivalent to AAV3B, which is at variance with the findings of Lisowski et al where transduction of Huh7 cells with AAVLK03 vectors was shown to be 43-fold higher than with AAV3B vectors [Lisowki, et al, Nature, 506: 382-386 (2014)].

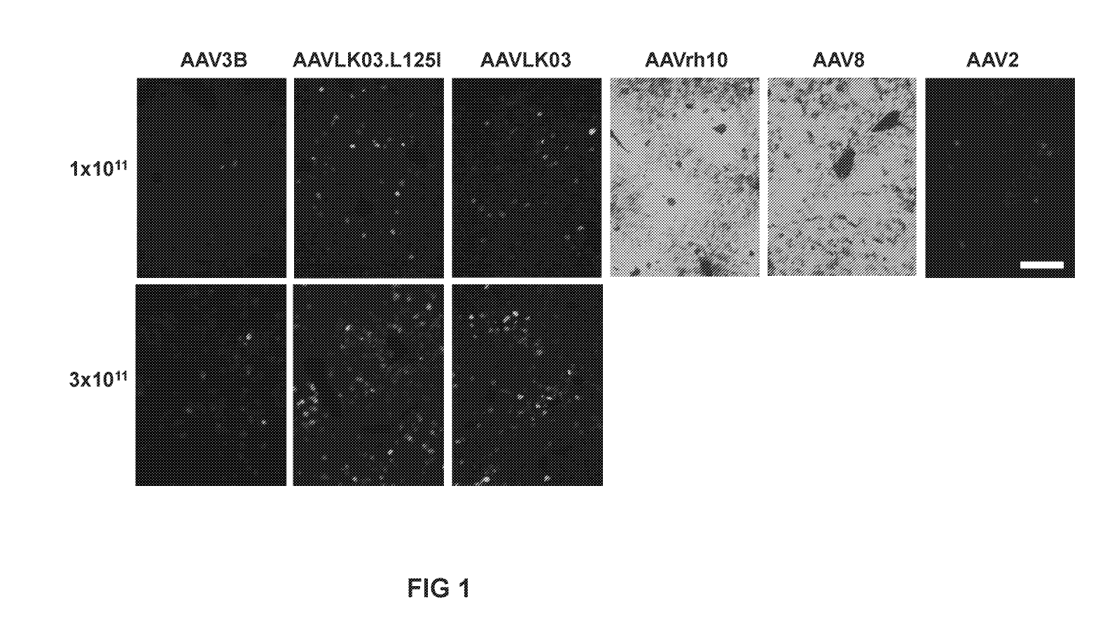

C57BL/6 mice were injected intravenously (IV) with different doses of vectors expressing green fluorescence protein (GFP) from the liver specific TBG promoter. Representative liver histology sections are presented in FIG. 1. At a dose of 1×10″ GC/mouse, the two clade E capsids AAVrh10 and AAV8 demonstrated very high transduction of hepatocytes (84% and 81%, respectively) while AAV3B, AAVLK03 and AAVLK03.L125I vectors poorly transduced mouse hepatocytes (0.1%, 3.9%, and 2.5%, respectively). These data are consistent with previous reports of high transduction in mouse liver in vivo of clade E vectors and low transduction of AAV3B and AAVLK03 [Wang, L., et al, Mol Ther, 18: 118-125 (2010); Lisowski, cited above].

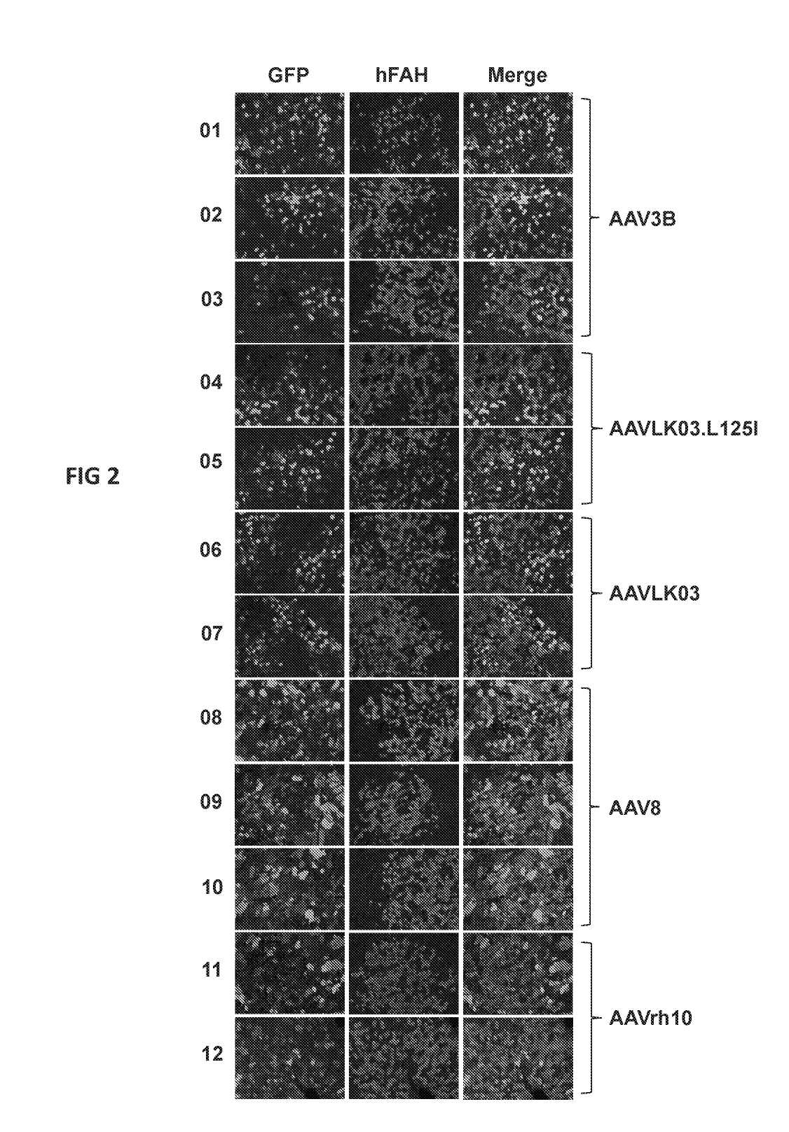

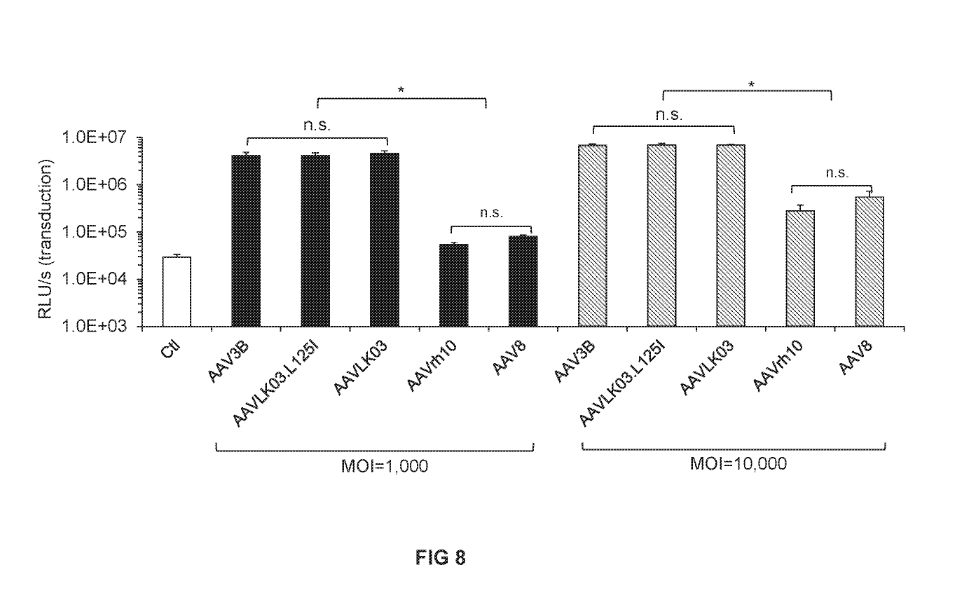

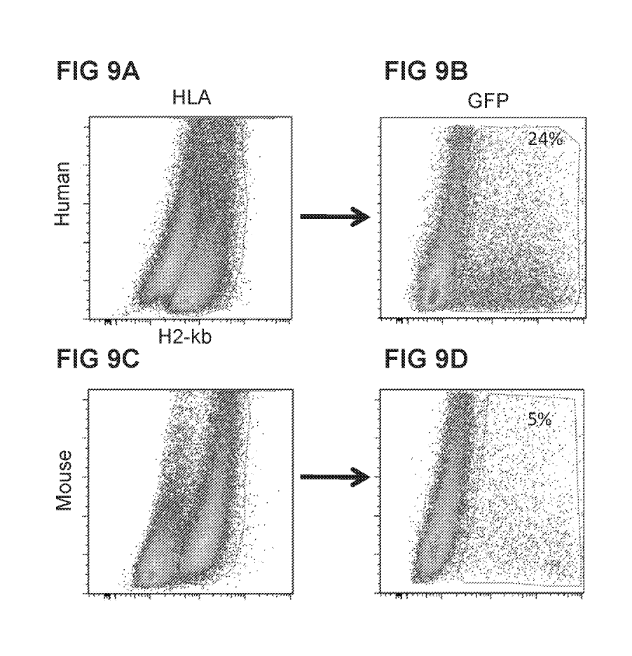

In an attempt to better model transduction of human liver, the Fah−/−/Rag−/−/I12rg−/− (FRG) mouse were utilized. In this model, in which the liver from this immune deficient mouse is partially repopulated with human hepatocytes (subsequently called the human liver xenograft model) [Bissig, K D, et al, Proc Natl Acad Sci USA, 104: 20507-20511; Azuma, H., et al, Nat Biotechnol, 25: 903-910 (2007); Bissig, K D, et al, J Clin Invest, 120: 924-930 (2010)]. Following IV injection of GFP expressing vector into the human liver xenograft model, liver was harvested and quantified transduction of endogenous mouse and human hepatocytes using two different approaches. The standard method is based on immunofluorescence analysis of liver tissue sections looking for co-localization of transgene expression with a cell specific marker for the engrafted human hepatocytes (i.e., human fumarylacetoacetase—hFAH). Morphometric analyses of these experiments revealed the following populations of cells: transduced human hepatocytes—GFP+hFAH+; non transduced human hepatocytes—GFP−hFAH+; transduced mouse cells—GFP+hFAH−, and non-transduced mouse hepatocytes—GFP−hFAH−. FIG. 2 presents representative fluorescent micrographs of liver harvested from xenograft mice 3 weeks after injection with 3×10¹¹ GC of AAV.TBG.GFP. In this analysis, green represents GFP expressing cells, red represents human FAH expressing cells and yellow represents cells expressing both markers. The remaining part of each liver was subjected to a second method for quantitating transduction based on flow cytometric analysis of single cell suspensions of hepatocytes released following perfusion with collagenase and staining with antibodies for mouse (H2-kb) and human (HLA) cells (FIG. 9). Transduction efficiencies were measured by co-localization of GFP with the cell specific markers. A summary of mouse xenograft studies in terms of transduction efficiencies using both methods is provided in Table 1.

TABLE 1
Differential transduction of human and mouse hepatocytes by AAV vectors^{a}

| | | % Transduction | | % Transduction | |
|---|---|---|---|---|---|
| | | (FACS)^{b} | | (Morphometric)^{c} | |
| Vector | Mouse | Human (MFI) | Mouse(MFI) | Human | Mouse |
| AAV3B | 01 | 26 (1.5E+5) | 6 (1.3E+5) | 21 ± 7 | 6 ± 2 |
| AAV3B | 02 | 19 (8.3E+4) | 10 (1.3E+5) | 31 ± 11 | 12 ± 7 |
| AAV3B | 03 | 24 (6.1E+4) | 6 (1.0E+5) | 17 ± 6 | 1 ± 0.4 |
| AAVLK03.L125I | 04 | 28 (5.3E+4) | 8 (9.0E+4) | 38 ± 17 | 13 ± 6 |
| AAVLK03.L125I | 05 | 28 (7.1E+4) | 12 (1.1E+5) | 36 ± 11 | 9 ± 3 |
| AAVLK03 | 06 | 30 (4.8E+4) | 15 (6.9E+4) | 31 ± 8 | 5 ± 3 |
| AAVLK03 | 07 | 32 (1.3E+5) | 17 (5.1E+4) | 17 ± 7 | 6 ± 3 |
| AAV8 | 08 | n/a | n/a | 16 ± 9 | 16 ± 4 |
| AAV8 | 09 | 61 (7.3E+4) | 42 (1.3E+5) | 25 ± 10 | 27 ± 4 |
| AAV8 | 10 | 32 (6.7E+4) | 28 (6.3E+4) | 39 ± 10 | 51 ± 7 |
| AAVrh10 | 11 | 23 (4.1E+4) | 14 (4.0E+4) | 19 ± 11 | 23 ± 4 |
| AAVrh10 | 12 | 58 (6.5E+4) | 27 (8.5E+4) | 38 ± 14 | 42 ± 12 |

^{a}Differential transduction of human and mouse hepatocytes was evaluated by flow cytometric analysis of isolated hepatocytes (FACS) or by sectioning whole livers (Morphometry).
^{b}For FACS, the transduced GFP positive subset and the mean fluorescent intensity (MFI) among the human or mouse hepatocytes is presented.
^{c}For morphometric analysis, individual liver sections were stained with an anti-human FAH antibody to differentiate human and mouse cells. The percent area (pixels) of GFP expression among human (GFP+/hFAH+) or mouse cells (GFP+/hFAH−) is presented along with standard deviation. n/a data was not available.

There was excellent correlation of human hepatocyte transduction with AAV3B and AAVLK03 vectors when comparing measures of transduction efficiency using the two different analytical methods. The average transduction efficiencies of human hepatocytes were as follows (% transduction by flow/% transduction by histology): AAV3B—23/23; AAVLK03 and AAVLK03.L125I—30/31; AAV8—47/27; and AAVrh10—41/29. High correlation between the two methods of quantitation was noted with mouse hepatocytes with the exception of some animals receiving Clade E vectors where histological analyses yielded higher estimates of transduction for reasons that are unclear but could relate to gating parameters. The relatively high level of transduction of human hepatocytes that was achieved with AAV8 is not consistent with the findings of Lisowski et al, who claimed that transduction of human hepatocytes with AAV8 was reduced 20-fold relative to AAVLK03 using the same human liver xenograft model and the same method of histochemical quantitation of GFP 25. Lisowski et al also claimed that AAVLK03 is significantly more efficient than AAV3B in the human liver xenograft model based on luciferase imaging; this is at variance with these studies that demonstrated equivalent transduction between these two vectors.

Male rhesus macaques were injected IV with the AAVrh10, AAV3B, AAVLK03 and AAVLK03.L125I vectors expressing GFP (N=2/vector) and 7-10 days later were necropsied and tissues were evaluated for expression and distribution of vector. One animal was infused with an AAV2-based vector to provide context for earlier pre-clinical and clinical studies when this capsid was the only one available for in vivo studies. Animals were pre-screened to assure they did not have pre-existing neutralizing antibodies (NAb) to the capsid of the vector that they received. Previously published data from AAV8.TBG.GFP injected animals (N=2, RQ8082 and RQ8083) are included for comparison [Wang, L., et al, Hum Gene Ther, 22: 1389-1401 (2011)]. All animals tolerated vector infusion without any clinical sequalae or abnormalities in blood hematology or chemistry (data not shown). A summary of the macaque studies is provided in Table 2.

TABLE 2
Summary of Gene Transfer in Macaques After systemic Vector Administration.

| | | | | | | | | | |
|---|---|---|---|---|---|---|---|---|---|
| AAV3B | RQ9759 | <1:5 | ++ | 25.6 ± 5.6 | 1.02 ±0.27 | 0.73 | 17.1 | 9.6 | 0.56 |
| | RQ9831 | <1:5 | ++ | 24.4 ± 17.2 | 1.16 ± 0.19 | 0.65 | 13.1 | 19.6 | 1.50 |
| AAVLK03.L125I | RQ8982 | <1:5 | +++ | 17.8 ± 9.2 | 0.66 ± 0.14 | 0.49 | 7.0 | 30.9 | 4.4 |
| | RQ9284 | <1:5 | +++ | 3.8 ± 1.8 | 0.51 ± 0.09 | 0.12 | 1.2 | 31.0 | 25.8 |
| AAVLK03 | RQ9828 | <1:5 | ++ | 15.2 ± 9.0 | 0.43 ± 0.07 | 0.18 | 0.5 | 34.0 | 68.0 |
| | RQ9837 | 1:20 | ++ | 0.1 ± 0.1 | 0.31 ± 0.04 | 0 | 0.003 | 25.3 | 8433.3 |
| AAVrh10 | 090-0266 | <1:5 | + | 14.5 ± 10.1 | 0.47 ± 0.11 | 0.18 | 9.6 | 7.1 | 0.74 |
| | 090-0283 | <1:5 | +/− | 15.8 ± 7.0 | 0.58 ± 0.21 | 0.45 | 20.1 | 9.4 | 0.47 |
| AAV8 | RQ8082 | <1:5 | + | 23.0 ± 17.6 | 0.79 ± 0.26 | 0.45 | 26.2 | 2.8 | 0.11 |
| | RQ8083 | <1:5 | − | 20.5 ± 12.7 | 0.92 ± 0.24 | 0.47 | 29.4 | 3.0 | 0.10 |
| AAV2 | 03D313 | <1:5 | ++ | 1.6 ± 1.8 | 0.35 ± 0.04 | 0.12 | 28.8 | 28.5 | 0.99 |

GFP, green fluorescent protein; NAb, neutralizing antibody.
^{a}Detection of capsid protein in spleen by immunofluorescence.
^{b}Measured as the percentage of GFP-positive area per liver section regardless of brightness. Ten sections per animal were analyzed.
^{c}Determined as the total intensity of each image with background level subtracted (see Material and Methods). Ten sections per animal were analyzed.
^{d}Determined by an ELISA on liver lysate from the right lobe of each monkey.
^{e}Determined by qPCR of transgene GFP, according to a standard curve generated with linearized plasmid DNA pAAV.CMV.GFP. The limit of detection is 7 × 10⁻⁵ copies/diploid genome

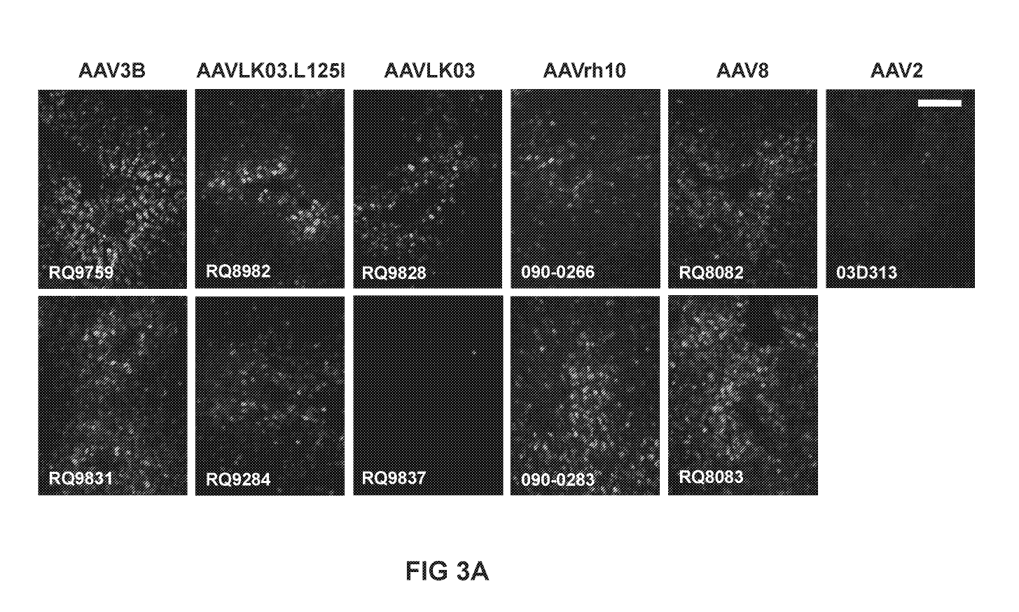

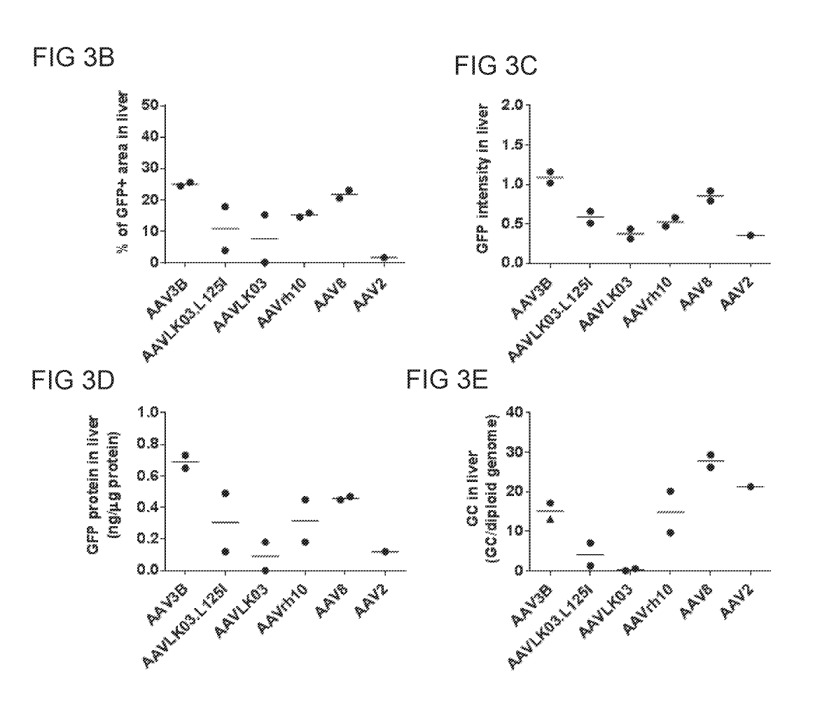

Liver tissue sections were visualized for transduction by fluorescence microscopy (FIG. 3A) and quantified for transgene expression by measuring % transduction (surface area of GFP fluorescence within the section—FIG. 3B) and GFP intensity (FIG. 3C). Liver homogenates were also analyzed by ELISA for GFP protein (FIG. 3D). Total vector genomes were measured by qPCR (FIG. 3E). The relative efficacy of transduction and gene transfer varied between capsids but in most cases was consistent between the 2 animals within a group. The hierarchy of performance was the same independent of how it was measured: AAV3B>AAV8>AAVrh10>AAVLK03=AAVLK03.L125I>AAV2. The efficiency of transduction was in excess of 20% of hepatocytes for both AAV3B and AAV8 with vector genomes in excess of 10 copies/diploid genomes for AAVrh10, AAV8 and AAV3B. One animal within the AAVLK03 group demonstrated virtually no detectable transduction or gene transfer. It was subsequently learned that this macaque seroconverted to AAVLK03 between the time of screening and dosing (i.e., NAb <1:5 6 weeks prior and 1:20 at time of dosing). Eliminating this animal from the analyses does not change the conclusions. This finding does reinforce the impact that pre-existing immunity can have on efficacy of liver gene therapy; previous studies with AAV8 in macaques demonstrated a substantial reduction in transduction at titers of NAb in excess of 1:10 which appears to be relevant to vectors of the AAV3 related family [Wang, L., et al, Hum Gene Ther, 22: 1389-1401 (2011)].

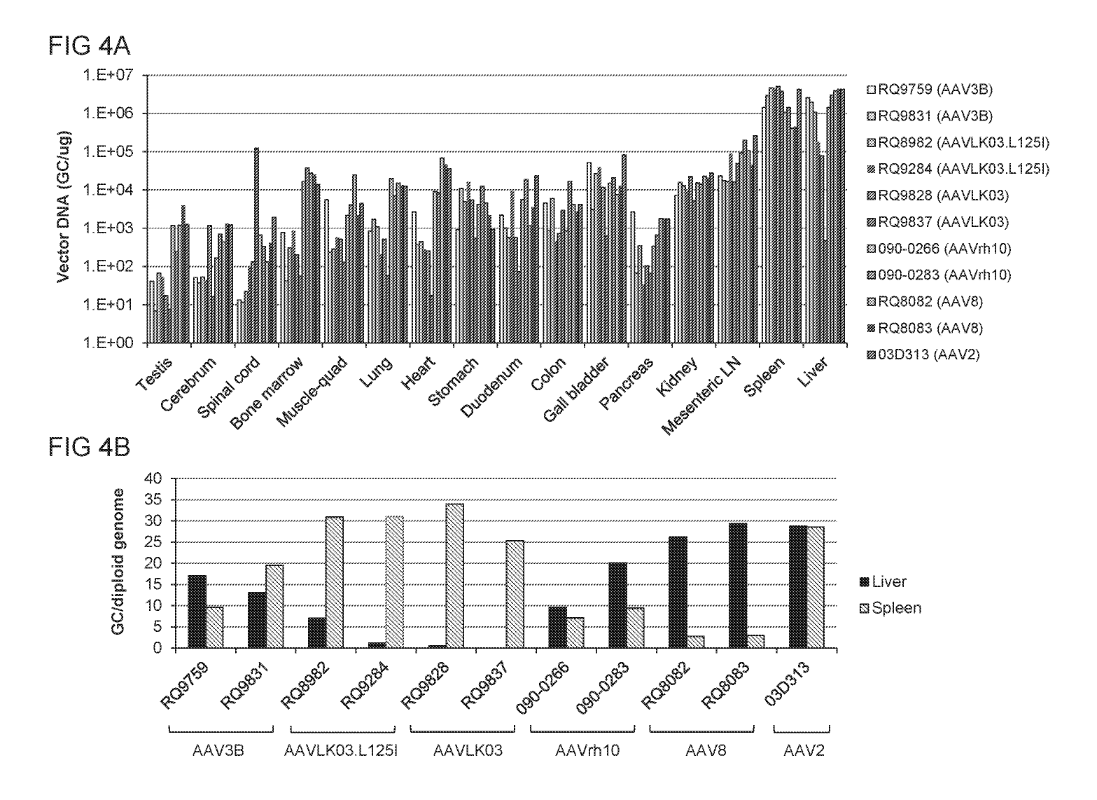

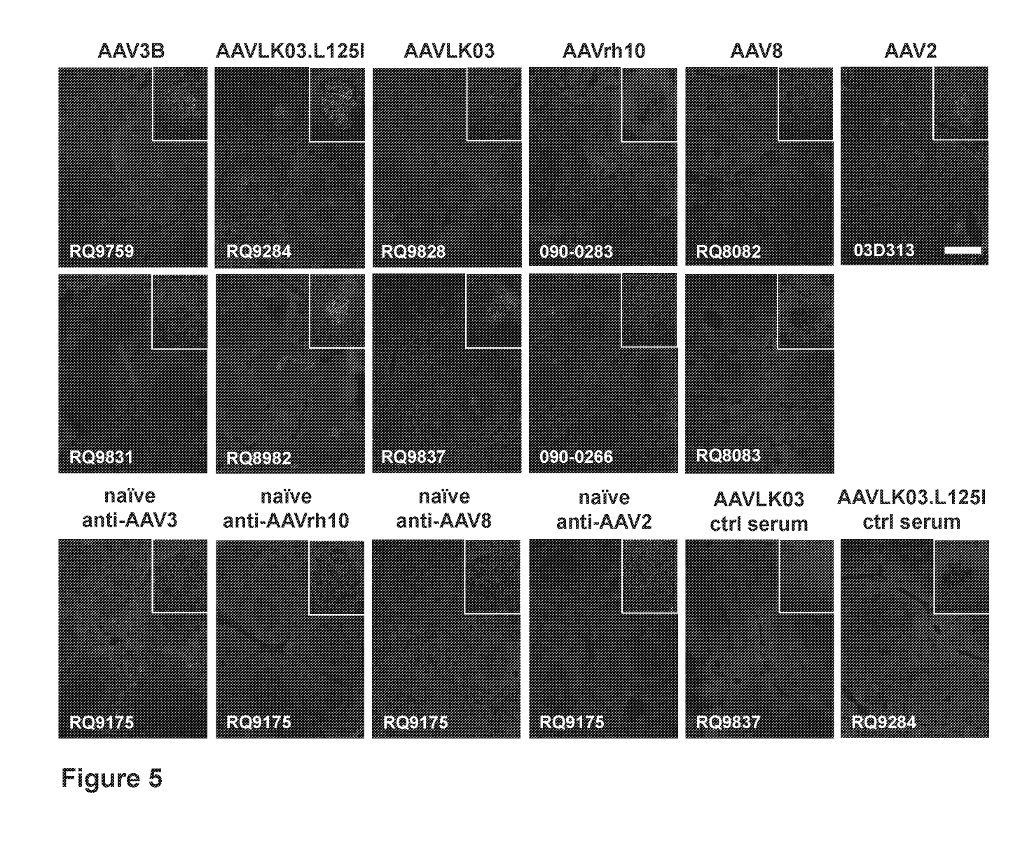

A more extensive analysis of tissues for bio-distribution of vector genomes was conducted (FIG. 4A). The data were virtually indistinguishable between the two clade E based vectors—AAV8 and AAVrh10—as well as the one animal who received an AAV2 vector. The profiles of vector distribution were also indistinguishable between the AAV3 related vectors (i.e., AAV3B, AAVLK03 and AAVLK03.L125I) although there were substantial differences between clade E/AAV2 vectors and AAV3 related vectors. With all vectors, liver and spleen harbored the highest level of vector although substantially more vector was directed to spleen from the AAV3 related vectors. Furthermore most other tissues contained higher levels of clade E vectors than the AAV3 related vectors. These clade specific differences in liver and spleen vector distribution is highlighted in FIG. 4B; the ratio of liver to spleen vector genomes was 5.7 for clade E vectors (range 1.3 to 9.8) and 0.5 for AAV3 related vectors (range 0.02 to 1.8, excluding RQ9837). Spleen tissue was further analyzed for presence of capsid protein by immunofluorescence with capsid specific antibodies (FIG. 5). Substantial quantities of capsid localized to splenic germinal centers following injection of the AAV3 family of vectors. Interestingly, this was not observed in spleen tissue from animals injected with clade E vectors. Within the AAV3 family, vector genomes and germinal center capsid protein was consistently higher with the AAVLK03 and AAVLK03.L125I as compared with AAV3B.

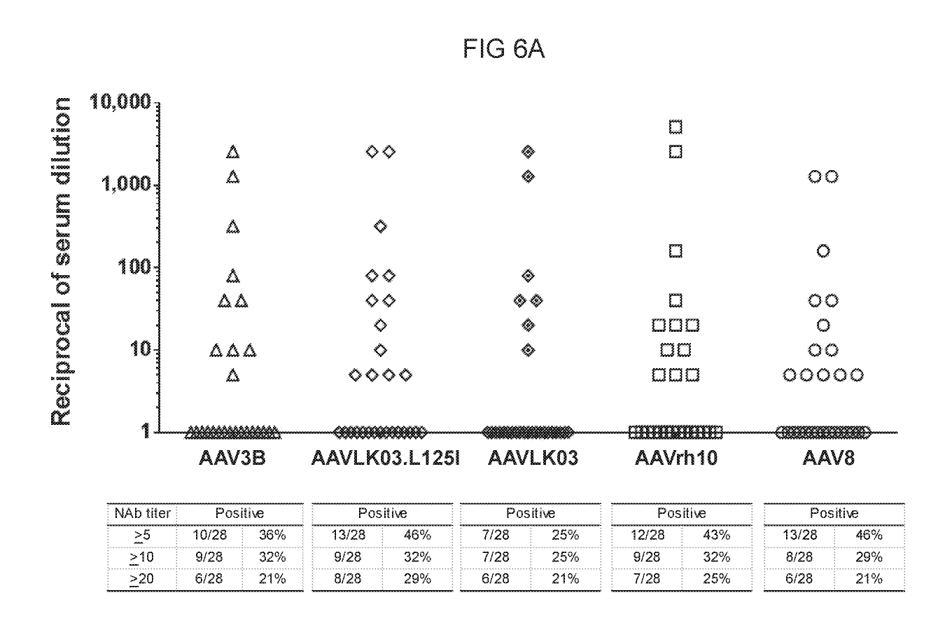

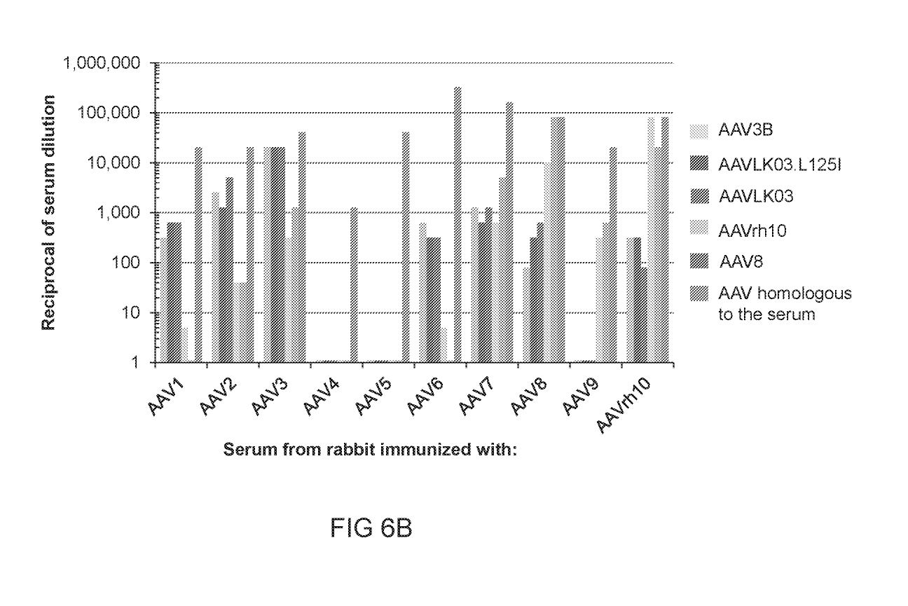

A potential impediment to successful liver gene therapy is antibody mediated inhibition of in vivo transduction. NAbs can form from natural AAV infections or from a previous AAV treatment. Serum from 28 healthy subjects from North America was surveyed for NAbs to the clade E and AAV3 family of vectors evaluated in this study (FIG. 6A). In this cohort there was essentially no difference in the prevalence of NAb titers greater than 1:10 which is the threshold previously shown for AAV8 was associated with substantial reductions in gene transfer in macaques [Wang, L, et al, Hum Gene Ther, 22: 1389-1401 (2011)].

These vectors were also evaluated for cross neutralization with sera generated in rabbits to the individual capsids. FIG. 6B presents the ability of sera generated to AAV1—AAV9 and AAVrh10 to neutralize the clade E and AAV3 related vectors that are the subject of this study. As expected there was a high degree of cross neutralization within the clade E vectors as well as within the AAV3 family of vectors although neutralization was substantially diminished when evaluated across clades/families. For example, sera generated to AAV8 neutralized the AAV3 family of vectors at titers that were reduced three logs compared to titer achieved against itself. A similar reduction in neutralizing titers to AAV3 related vectors was observed with sera generated to the other AAVs currently used in clinical trials (AAV9 and AAVrh10) as compared to the effectiveness of the sera to neutralize the capsid to which the sera were generated.

Data generated in the humanized xenograft mice indicated that vectors based on AAVLK03 and AAVLK03.L125I are indistinguishable from AAV3B, which is not surprising considering the high degree of homology between these capsids. However, the transduction of the monkey liver by LK03 type vectors is considerably lower than AAV3B, and much more of LK03 vectors are directed to spleens in monkeys. This study is the first to conduct head-to-head comparisons of these putative hepatotropic vectors in nonhuman primates. Studies in mice did not predict the outcome in primates especially with AAV3 related vectors. However, results in the human liver xenograft model, as generated in this laboratory, did predict the general transduction efficiencies in the nonhuman primates across all capsids.

### Example 3—Purification of AAV Vectors Having a SPAKFA Residue

This study shows that an epitope within the AAV3B capsid enables its binding to a sepharose high performance column (e.g., a cross-linked 6% agarose) which utilizes a 14kD fragment from a single chain llama antibody expressed in yeast, commercially available from GE Healthcare LifeSciences as an AVB Sepharose. The studies demonstrate that this epitope may be engineered into other AAV capsids which naturally lack this epitope for purification, without any deleterious reduction in transduction efficiencies and without any undesirable alteration in tropism.

### Materials and Methods

A. Plasmids.

Constructs pAAV2/8, pAAV2/rh.64R1, pAAV2/9 and pAAV2/3B expressing the AAV8, rh.64R1, AAV9, and AAV3B capsid protein respective were used. Mutagenesis of these plasmids was carried out with QuikChange™ Lightning Site-Directed Mutagenesis Kit (Agilent Technologies, CA), following the manual's instructions. The primers for the mutagenesis were:

SEQ ID NO: 7:
5′-ATCCTCCGACCACCTTCAGCCCTGCCAAGTTTGCTTCTTTCATCACG
CAATA-3′
and
SEQ ID NO: 8:
5′-TATTGCGTGATGAAAAAGCAAACTTGGCAGGGCTGAAGGTGGTCGGA
GGAT-3′ for pVAA2/8 (NQSKLN→SPAKFA),
SEQ ID NO: 9:
5′-ATCCTCCAACAGCGTTCAGCCCTGCCAAGTTTG-CTTCTTTCATCAC
GCAGTA-3′
and
SEQ ID NO: 10:
5′-TACTGCGTGATGAAAGAAGCAAACTTGGCAG-GGCTGAACGCTGTTG
GAGGAT-3′ for pAAV2/rh.64R1 (NQAKLN→SPAKFA),
SEQ ID NO: 11:
5′-ATCCTCCAACGGCCTTCAGCC-CTGCCAAGTTTGCTTCTTTCATCAC
CCAGTA-3′
and
SEQ ID NO: 12:
5′-TACTGGGTGATGAAAGAAGCAAACTTGGC-AGGGCTGAAGGCCGTTG
GAGGAT-3′ for pAAV2/9 (NKDKLN→SPAKFA),
SEQ ID NO: 13:
5′-ATCCTCCGACGACTTTCAACAAGGACAAGC-TGAACTCATTTATCAC
TCAGTA-3′
and
SEQ ID NO: 14:
5′-TACTGAGT-GATAAATGAGTTCAGCTTGTCCTTGTTGAAAGTCGTCG
GAGGAT-3′ for pAAV2/3B (SPAKFA→NKDKLN).

B. Vectors.

Purified vector preparations AAV2/8.CMV.ffluciferase.SV40 AAV2/rh.64R1.CMV.PI.EGFP.WPRE.bGH, AAV2/AAVhu37.TBG.EGFP.bGH and AAV2/AAVrh10.CMV.PI.Cre.RBG, were produced and titrated by Penn Vector Core as previously described [Lock, M, et al. (2010). Rapid, Simple, and Versatile Manufacturing of Recombinant Adeno-Associated Viral Vectors at Scale. Human Gene Therapy 21: 1259-1271]. Briefly, one cell stack (Corning, N.Y.) of HEK293 cells was transfected with triple-plasmid cocktail by Polyethylenimine (PEI) when the cell confluency reached around 85%. Culture supernatant was harvested 5 days post transfection and digested with Turbonuclease (Accelagen, CA). NaCl was added to a concentration of 0.5M and the treated supernatant was then concentrated with Tangential Flow Filtration (TFF). Concentration was followed by iodixanol density gradient ultracentrifugation and final formulation by buffer-exchange through Amicon® Ultra-15 (EMD Millipore, MA) into DPBS (Dulbecco's Phosphate-Buffered Saline without calcium and magnesium, 1×, Mediatech, VA) with 35 mM NaCl. Glycerol was added to 5% (v/v) and the vectors were stored at −80° C. until use. For titration, real-time PCR with Taqman reagents (Applied Biosystems, Life Technologies, CA) was performed targeting RBG, bGH and SV40 polyadenylation sequences respectively. AAV2/3B.CB7.CI.ffluciferase.RBG was made the same way, except that at the TFF step, AVB.A buffer (Tris pH 7.5, 20 mM, NaCl 0.4 M) was used for buffer-exchange. The retentate was then stored at 4° C. and 0.22 μm-filtered before application to the AVB column.

For the vectors used for the wild type—SPAKFA mutant comparison, each wild type capsid and its mutants were made in parallel from one 15-cm plate, using a version of the protocol described above but scaled down proportionally according to the culture area of the plate. Culture supernatant was treated with Turbonuclease and then stored at −20° C. Before application to the AVB column, the supernatant was clarified at 47,360× g and 4° C. for 30 minutes followed by 0.22 μm filtration. The transgene cassette for these vectors was CB7.CI.ffluciferase.RBG.

C. Chromatography.

An AKTAFPLC system (GE Healthcare Life Sciences, NJ) was used for all binding studies. The HiTrap column (1 mL) used was prepacked with AVB Sepharose™ High Performance resin (GE Healthcare Life Sciences, NJ). AAV vectors were reconstituted in AVB.A buffer and loaded onto a column equilibrated in the same buffer. The column was washed with 6 mL of AVB.A buffer and 5 mL of AVB.C buffer (Tris pH 7.5, 1 M NaCl) and then eluted with 3 mL of AVB.B buffer (20 mM sodium citrate, pH 2.5, 0.4 M NaCl). The eluted peak fractions were immediately neutralized with 1/10× volume of BTP buffer (0.2 M Bis tris-propane, pH 10). The flow rate was 0.7 mL/min (109 cm/hour). For testing the affinity of AAV vectors for the AVB resin, equal genome copy numbers (GC) of purified AAV8, AAVrh10 and AAVhu37 vectors were mixed together before loading. For rh.64R1, the load consisted of an equal amount (GC) of AAVrh10 and AAVrh64R1 in AVB.A buffer. For AAV3B, the clarified AAV3B product was loaded directly onto the AVB column. For the comparison of AAV vectors and their SPAKFA mutants, 9.5 mL of the clarified product was loaded onto the AVB column at 0.7 mL/min, followed by washing with 8 ml of DPBS and 5 ml of AVB.C at 1 mL/min, and then eluted with 4 mL of AVB.B at 0.25 mL/min. The eluate was immediately neutralized as above.

D. In Vitro Infectivity Assay.

Huh7 cells were seeded in 96-well plates at a density of 5e4 cells/well. The cells were then infected with AAV vectors carrying the CB7.CI.ffluciferase.RBG transgene cassette 48 hours after seeding. Three days post-infection, luciferase activity was measured using a Clarity luminometer (BioTek, VT).

E. Sequence Alignment and Structure Analysis.

Sequence alignments were done with the ClustalW algorithm by the AlignX component of Vector NTI Advance 11.0 (Invitrogen, CA). The protein sequences were: AAV1 (accession: NP_049542), AAV2 (accession: YP_680426), AAV3 (accession: NP_043941), AAV3B (accession: AAB95452), AAV5 (accession: YP_068409), AAVrh10 (accession: AA088201), AAVhu37 (accession: AAS99285), AAV8 (accession: YP_077180), rh.64R1 (accession: ACB55316), AAV9 (accession: AAS99264). Structure analysis was performed with the Chimera program [Pettersen, E F, et al. (2004). J Comput Chem 25: 1605-1612; Sanner, M F, et al, (1996). Biopolymers 38: 305-320] and the AAV8 capsid structure (PDB: 2QA0) [Nam, H J, et al, (2007). Structure of adeno-associated virus serotype 8, a gene therapy vector. J Virol 81: 12260-12271].

### Results

F. The Affinity of AVB Resin for AAV Serotypes Varied Significantly.

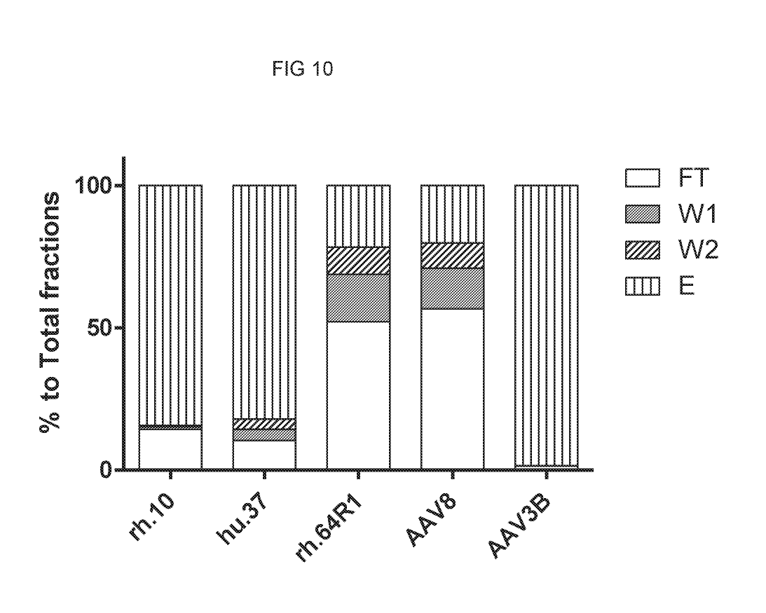

To test the affinity of AVB resin for AAV8, AAVrh64R1 and AAVhu37 serotypes, AAV vector preparations were mixed together and the AAVrh10 serotype was added as an internal positive control. This mixing of preparations was performed in order to minimize variations during chromatography. Because of limited choices of real-time PCR probes, two types of vector mixes were made, AAV8+AAVhu37+AAVrh10 and AAVrh64R1+AAVrh10, and run on the AVB affinity column. The AAVrh10 vector genome distribution among the different fractions collected was very similar between the two runs (data not shown), so the average of the two runs was used for reporting the AAVrh10 data. As show in (FIG. 10), 84% of the loaded AAVrh10 vector genome was present in the elution fraction. The affinity of the AAVhu37 vector was similar to AAVrh10, with 82% in the elution fraction. On the contrary, both AAV8 and rh.64R1 vectors bound AVB resin poorly, with only 20% and 22% in the elution fraction, respectively. The affinity of AAV3B for AVB resin was remarkable, with 98% of vector genomes recovered in the elution fraction.

G. Sequence Alignment and Structure Analysis Showed that the Amino Acid Region 665-670 (AAV8 VP1 Numbering) was the Most Diverse Region on the Capsid Surface Between the High AVB-Affinity AAV Serotypes, AAV3B, AAVrh10 and AAVhu37, and the Low Affinity Serotypes, AAV8 and Rh.64R1.

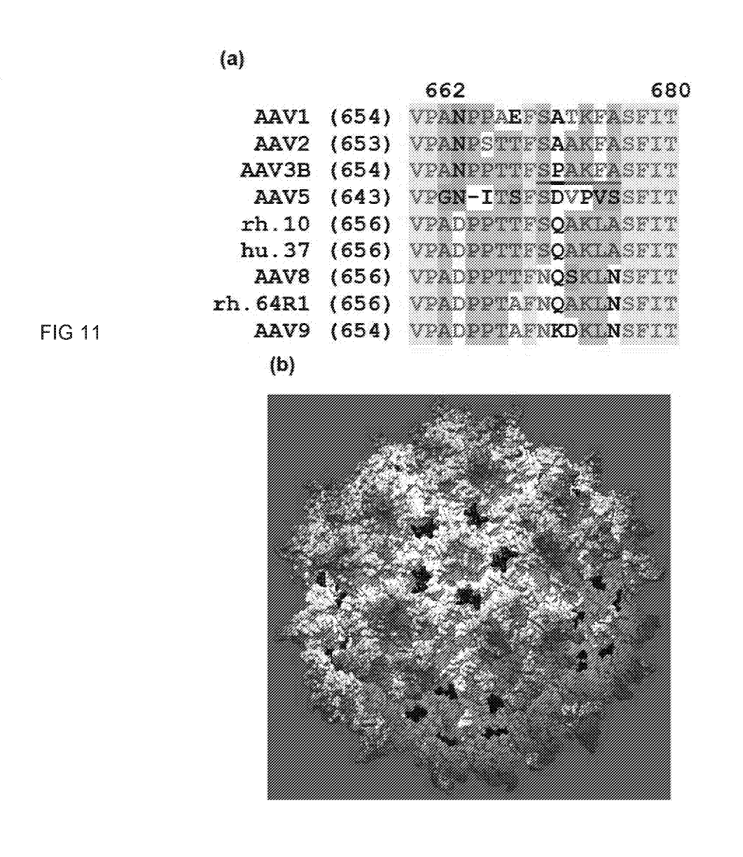

Among the residues exposed on the surface of the AAV8 capsid (PDB accession number: 2QA0 [Nam, H J, et al, J Virol, 81: 12260-12271 (2007)]), the following twenty six residues are identical between AAVrh10 and AAVhu37 serotypes but different from AAV8 (numbering format: AAV8 residue-AAV8 VP1 numbering (SEQ ID NO:1)-AAVrh10/AAVhu37 residue): A269S, T453S, N459G, T462Q, G464L, T472N, A474S, N475A, T495L, G496S, A507G, N517D, I542V, N549G, A551G, A555V, D559S, E578Q, I581V, Q594I, I595V, N665S, S667A, N670A, S712N, V722T. Among the 26 residues, only residue 665 (AAV8 VP1 numbering, SEQ ID NO:1), is identical among AAV1, AAV2, AAV3B, AAV5, AAVrh10 and AAVhu37. As shown in (FIG. 11A), all the poor affinity AAV serotypes (AAV8, rh.64R1 and AAV9) have an Asn residue at this position while the high affinity serotypes have Ser. The 665 residue locates in a small variable patch (665-670, AAV8 VP1 numbering) of the AAV capsid. The entire patch is exposed at the capsid surface, near the pore region (FIG. 11B) and this whole epitope was therefore selected for swapping experiments. Because the affinity of the AAV3B serotype for AVB resin is very good, the SPAKFA epitope from AAV3B was selected to swap into the AAV8, rh.64R1 and AAV9 serotypes using site-specific mutagenesis. The resulting mutants were denoted as AAVx-SPAKFA. As a control, a reverse swap mutant was made where the corresponding epitope of AAV9 (NKDKLN, SEQ ID NO: 2) was swapped into the AAV3B capsid; the resulting mutant was named AAV3B-NKDKLN. The vector production yield of the SPAKFA epitope mutants was 81% (AAV8), 82% (rh.64R1) and 137% (AAV9) of their wild-type counterparts. The yield of AAV3B-NKDKLN was 28% of AAV3B.

H. SPAKFA Epitope Exchange Greatly Improved the AVB-Affinity of AAV8, Rh.64R1 and AAV9 Serotypes.

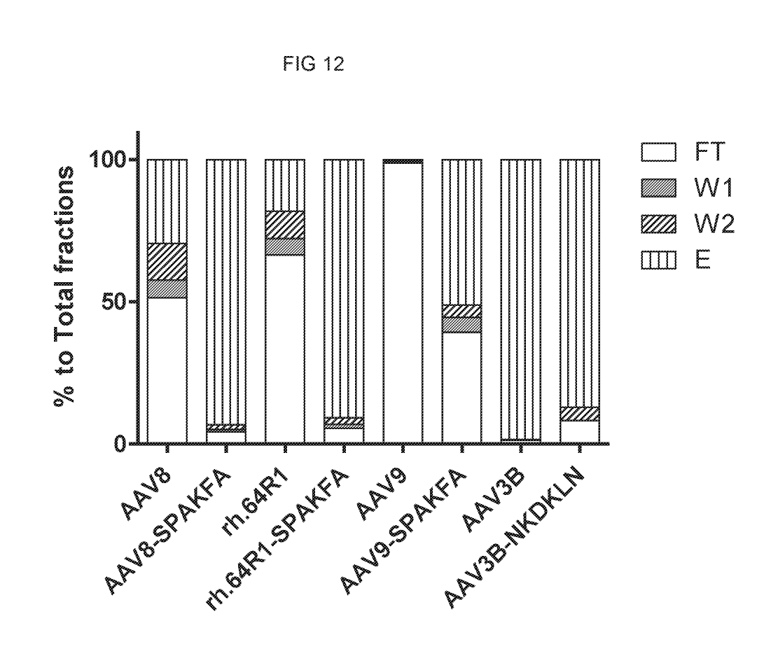

As shown in (FIG. 12), after SPAKFA substitution, clear improvement in the affinity of AAV8, rh.64R1 and AAV9 serotypes was shown, with the percentage recovery of loaded vector in the elution fraction rising from 30%, 18%, and 0.6% of total fractions to 93%, 91% and 51%, respectively. In contrast, when the NKDKLN epitope of AAV9 was swapped into AAV3B, the elution fraction yield decreased from 98% to 87%, and the fractions of flow-through (FT), wash 1 (W1) and wash 2 (W2), rose from 1.3% to 8.2%, 0.0% to 0.1%, and 0.3% to 4.6% respectively. The majority of AAV3B-NKDKLN was still in the elution fraction however, indicating the existence of other epitope(s) which are involved in binding to AVB resin.

I. The Infectivity of AAV Vectors Containing SPAKFA Substitutions were Unaffected by the SPAKFA Swapping.

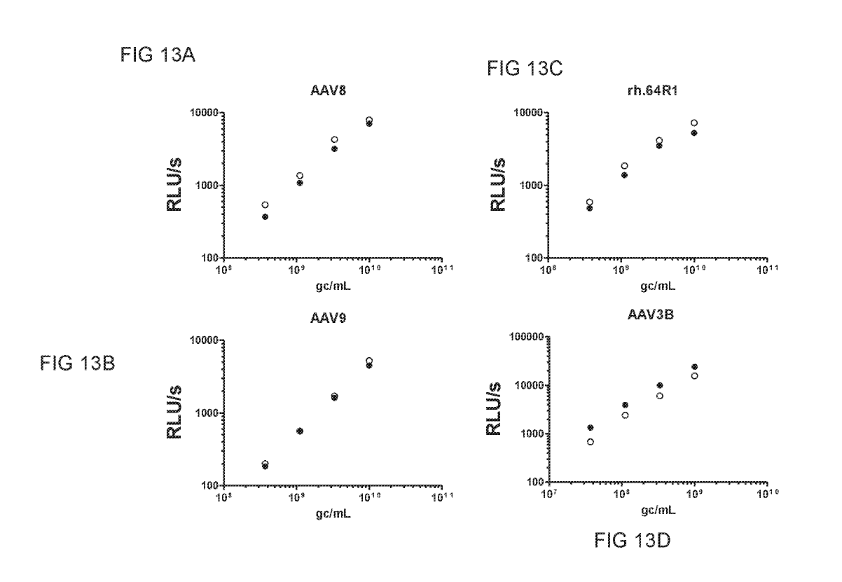

One key question was whether the epitope swapping performed impaired the potency of the recipient vector. To address this question, an in vitro infectivity assay was performed with the epitope substitution mutants in Huh7 cells. A range of vector concentrations were used for infection in order to avoid the possible saturation of transduction pathways at high multiplicity of infection (M.O.I.). For AAV3B and AAV3B-NKDKLN, the vector concentration used for infection was 1 log lower than for the other AAV vectors due to the very high infectivity of the AAV3B serotype for Huh7 cells. As shown in (FIG. 13), the infectivity of the SPAKFA mutants was comparable to that of the wild-type AAV vectors. The infectivity of AAV3B-NKDKLN was 59% of AAV3B.

A simple, efficient, generic and easily scalable purification protocol which can be used for all AAV serotypes is highly desirable. Affinity resins such as AVB will likely play an important role in enabling such a process as recently demonstrated in a study by Mietzsch and colleagues, [(2014) Human Gene Therapy 25: 212-222] in which 10 serotypes (AAV1-8, AAVrh10 and AAV12) were purified in a single step from clarified crude lysate using the AVB resin. However, this example shows that although AAV8, rh.64R1, AAVhu37, AAVrh10 and AAV3B can be captured by AVB resin, the affinity of the resin for these different serotypes is very different, with AAV3B having a strong affinity and AAV8 and rh.64R1 binding more poorly. While further optimization of buffers and flow rate can improve binding of AAV8 in our hands (data not shown), conditions and the resulting resin capacity are still not optimal for process scale-up.

The variation in AVB affinity for AAV serotypes AAVrh10, AAV8, AAVhu37 and rh.64R1 serotypes was intriguing since they all belong to Clade E and display a high degree of sequence similarity. By contrast, another serotype, AAV5, binds well to AVB but is distantly related to Clade E members. These observations led us to speculate that some subtle sequence differences may play a role in the different binding affinities of these serotypes to AVB. Sequence alignment and structure analysis of the VP3 capsid proteins of these serotypes led us to narrow in on residue 665. At this position, AAV8 and rh.64R1 are asparagine while AAVrh10, AAVhu37 and AAV5 are serine. Because the sequence patch around residue 665 is a small variable region, it was decided to swap the whole patch of AAV8, rh.64R1 and AAV9 with the patch (SPAKFA) from AAV3B. The clear improvement in affinity observed following these substitutions indicates that the SPAKFA sequence patch is an epitope of the AVB resin. Importantly, the substitutions did not affect the capsid fitness, in terms of yield and in vitro infectivity.

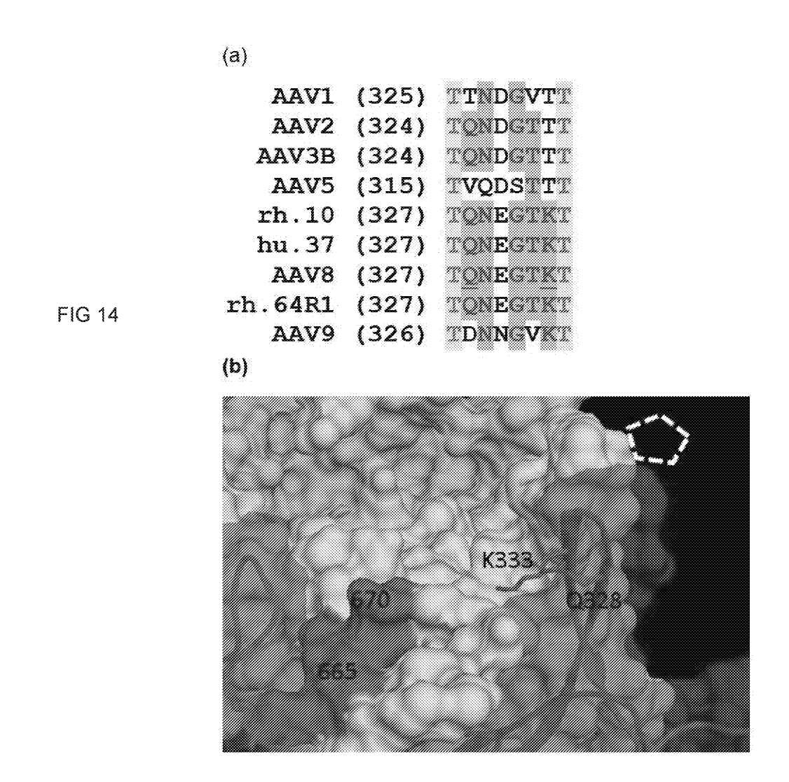

Another interesting observation was made when the corresponding sequence patch from the AAV9 serotype, NKDKLN (SEQ ID NO:2), was substituted in place of the SPAKFA epitope in the AAV3B capsid. While the affinity of the AAV3B-NKDKLN vector was apparently weakened, as evidenced by the appearance of the vector in the flow-through fraction, the majority still bound to the column. This result, in conjunction with the fact that substitution of the SPAKFA epitope into AAV9 did not produce the affinity observed with AAV3B, suggests there are other epitopes besides SPAKFA in the AAV3B VP3 amino acid sequence which contribute to AVB binding. One epitope candidate is the region containing residues 328-333 (FIG. 14). This region is at the outside surface of the pore wall, and is spatially close to the residues 665-670 region (based on numbering of SEQ ID NO:1). Residue 333 is especially close in spatial terms to the residues 665-670 region and for weak AVB binders such as AAV8, AAVrh64R1 and AAV9, this residue is Lysine, while in stronger binding serotypes such as AAV3B it is threonine. The hypothesis suggested by these observations is that the regions containing residues 665-670 and 328-333 both contribute to AVB binding, although residues 665-670 make the major contribution. The AVB binding data generated in this study in addition to the AAV3B-NKDLN data described above, support this hypothesis. Serotypes with high SPAKFA homology in the 665-670 region and a threonine residue at position 333 bind best to AVB (AAV3B, AAV1, AAV2 and AAV5). Serotypes with low SPAKFA homology and a lysine residue at position 333 bind poorly (AAV8, rh64R1 and AAV9). Intermediate cases such as serotypes rh10, hu37 and epitope-substituted mutants which contain SPAKFA but have lysine rather than threonine at position 333 (AAV8-SPAKFA and rh64R1-SPAKFA) do bind to AVB resin but less well than serotypes such as AAV3B. Further mutagenic analysis of the 328-333 region and confirmation of its role in AVB binding is complicated because it overlaps the coding sequences for the assembly-activation protein (AAP) in another reading frame [Sonntag, F, et al, (2010), Proc Natl Acad Sci USA, 107: 10220-10225].

The discovery of the SPAKFA-epitope can be useful in predicting whether AVB is a suitable resin for purification of some of the less commonly used AAV serotypes. For example, among the clade E members, rh.8, rh.43 and rh46 serotypes have sequences very similar to AAV8 at residues 665-670 and so their affinity for AVB will probably be low. On the other hand, rh.39, rh.20, rh.25, AAV10, bb.1, bb.2 and pi.2 serotypes are likely to bind well because their sequence in this region is identical (or very similar) to AAVrh10. Similarly, for many clade D members the 665-670 amino acid sequence is TPAKFA [SEQ ID NO: 15] and thus these serotypes are likely to display high affinity to AVB, while the AAVrh69 serotype is likely to bind poorly since the 665-667 amino acid sequence is NQAKLN [SEQ ID NO: 16].

Substitution of the SPAKFA epitope into the capsids of poor-affinity AAV serotypes such as AAV9 would permit for the use of AVB as a universal affinity chromatography resin for all AAV serotypes. In the studies presented here, yields and infectivity of epitope-substituted vectors were unaffected but the impact on tropism was not investigated since it was beyond the scope of this work. However, there are reports which show that the tropism of AAV8 vectors relates mainly to hyper-variable region VII (AAV8 549-564) and IX (AAV8 708-720) [Tenney, R M, et al (2014), Virology 454: 227-236], and/or the subloop 1 (AAV8 435-482) and subloop 4 (AAV8 574-643) [Shen, X, (2007) Molecular Therapy 15: 1955-1962] of the AAV8 capsid. Neutralizing epitope mapping data also supports the notion that the pore structure of AAV capsids and its nearby regions which are responsible for binding to AVB resin are not involved in cell transduction and therefore tropism. Neutralizing epitopes identified so far mainly locate around the 3-fold protrusion of the AAV capsid [Gurda, B L, et al. (2012). Journal of Virology 86: 7739-7751; Adachi, K, et al (2014). Nat Commun 5: 3075; Moskalenko, et al. (2000) Journal of Virology 74: 1761-1766; Wobus, C E, et al (2000). Journal of Virology 74: 9281-9293; Gurda, B L, et al. (2013). Journal of Virology 87: 9111-9124]. Indeed, for AAV2, switching the tip (RGNR) of the 3-fold protrusion resulted in dramatic changes in the tropism of the vector. Another relevant antibody study was performed with monoclonal mouse antibody 3C5 raised against AAV5. This antibody is not neutralizing [Harbison, C E, et al (2012). Journal of General Virology 93: 347-355] and one of its epitopes locates in the 665-670 region [Gurda et al, 2013, cited above]. This observation therefore suggests that antibody binding in this region does not affect cell transduction and by extension, tropism.

The ability to screen for AVB resin binding based upon the primary amino acid sequence, would greatly facilitate the process of selecting suitable AAV. For those serotypes where AVB resin binding is predicted to be poor, the substitution of the SPAKFA epitope may present a viable solution and enable the institution of a universal purification process for multiple serotypes.

All publications, patents, and patent applications cited in this application, and U.S. Provisional Patent Application No. 62/193,621, filed Jul. 17, 2015, are hereby incorporated by reference in their entireties as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications can be made thereto without departing from the spirit or scope of the appended claims.

TABLE
(Sequence Listing Free Text)

| SEQ ID NO | Free Text under <223> |
|---|---|
| 4 | AAVLK03 variant of AAV3 |
| 5 | variant of LK03 in which Leu at position 125 is |
| | substituted with an Ile |
| 6 | S663V + T492V modified AAV3B |
| 7 | NQSKLN to SPAKFA primer |
| 8 | pAAV2/8 (NQSKLN to SPAKFA) |
| 9 | pAAV2/rh.64R1 (NQAKLN to SPAKFA) primer |
| 10 | pAAV2/rh.64R1 (NQAKLN′SPAKFA) |
| 11 | pAAV2/9 (NKDKLN′SPAKFA) |
| 12 | pAAV2/9 (NKDKLN to SPAKFA) Primer 2 |
| 13 | pAAV2/3B (SPAKFA to NKDKLN) |
| 14 | pAAV2/3B (SPAKFA to NKDKLN) Primer 2 |
| 15 | Clade E epitope |
| 16 | Epitope |
| 17 | AAV1 epitope |
| 18 | AAV2 epitope |
| 19 | AAV5 epitope |
| 20 | AAVrh10 epitope |
| 21 | AAVhu37 epitope |
| 22 | AAVrh64R1 |
| 23 | AAV9 epitope |
| 24 | AAV capsid binding epitope |

The following information is provided for sequences containing free text under numeric identifier <223>.

1. A recombinant virus having a capsid with an engineered epitope comprising the amino acids SPAKFA (SEQ ID NO: 24), which is not present in the corresponding native AAV capsid, said capsid having packaged therein an expression cassette comprising an exogenous sequence encoding a gene product under control of regulatory sequences which direct expression thereof in a cell.

2. The recombinant virus according to claim 1, wherein the recombinant virus is a recombinant adeno-associated virus and the capsid is an adeno-associated virus capsid which comprises vp1 and vp3 capsid proteins, and optionally vp2 capsid proteins.

3. The recombinant virus according to claim 2, wherein the epitope is inserted in the region of amino acids 665 to 670 based on the numbering of the vp1 capsid of AAV8 [SEQ ID NO:3].

4. The recombinant virus according to claim 2, wherein the epitope is fused at the end of the vp2 or vp3 protein.

5. A method for purifying a recombinant virus, said method comprising purifying a recombinant virus of any of claims 1 to 4 using a solid support which comprises an antibody specific for the SPAKFA epitope.

6. The method according to claim 5, wherein the solid support is an affinity capture affinity resin.

7. A regimen for delivery of a gene product to a human patient, said regimen comprising (a) delivery of a first recombinant AAV vector comprising an expression cassette comprising an exogenous sequence encoding a gene product under control of regulatory sequences which direct expression thereof in a cell; and (b) delivery of a second recombinant AAV vector comprising an expression cassette comprising an exogenous sequence encoding a gene product under control of regulatory sequences which direct expression of the product in a cell, wherein the first recombinant AAV vector or the second AAV vector has an AAV3B capsid.

8. The regimen according to claim 7, wherein the other of the first or the second AAV vector has a capsid which is selected from Clade E.

9. The regimen according to claim 8, wherein the Clade E vector has a capsid selected from an AAV8 capsid or an AAVrh10 capsid.

10. The regimen according to claim 7, wherein the liver cells of the patient are targeted.

11. The regimen according to claim 7, wherein the first and/or the second AAV vector comprise liver-specific regulatory sequences.

12. The regimen according to claim 11, wherein the regulatory sequences comprise a liver-specific promoter.

13. The regimen according to claim 11, wherein the regulatory sequences comprise a constitutive promoter.

14. The regimen according to claim 7, wherein the first AAV is delivered to neonatal patients.

15. The regimen according to claim 7, wherein the second AAV is delivered following the neonatal stage.

16. The regimen according to claim 7, wherein the first AAV is delivered to proliferating cells.

17. The regimen according to claim 7, wherein the delivery of the first rAAV and the second rAAV are temporally separated by at least one month.

18. The regimen according to claim 17, wherein the delivery of the first rAAV and the second rAAV are temporally separately by at least three months.

19. The regimen according to claim 17, wherein the delivery of the first rAAV and the second rAAV are temporally separately by at least about 1 year to about 10 years.

20. The regimen according to claim 7, wherein the regimen further comprises delivery of at least a third AAV, wherein said third AAV has a capsid which differs from AAV3B.

21. The regimen according to claim 7, wherein the AAV3B capsid is selected from AAV3B, AAVLK03, and AAVLK031125.

22. The regimen according to claim 7, wherein the AAV3B capsid is AAV3B.

23. The regimen according to claim 7, wherein first and/or second rAAV are delivered via intravenous delivery.

24. A method for targeting human spleen cells which comprises delivering a recombinant AAV vector comprising an AAV3B capsid having packaged therein an expression cassette comprising an exogenous sequence encoding a gene product under control of regulatory sequences which direct expression thereof in a cell.

25. A method for targeting human hepatocytes in a patient having pre-existing immunity to a Clade E AAV, said method comprising delivering a recombinant AAV vector comprising an AAV3B capsid having packaged therein an expression cassette comprising an exogenous sequence encoding a gene product under control of regulatory sequences which direct expression thereof in a cell.

26. The method according to claim 25, wherein the Clade E AAV is selected from AAV8 or AAV rh10.

27. Use of a recombinant AAV vector comprising an AAV3B capsid having packaged therein an expression cassette comprising an exogenous sequence encoding a gene product under control of regulatory sequences which direct expression thereof in a cell in a method for targeting human spleen cells.

28. A recombinant AAV3B vector comprising an AAV3B capsid having packaged therein an expression cassette comprising an exogenous sequence encoding a gene product under control of regulatory sequences which direct expression thereof in a cell, said AAV3B vector being suitable in a method for targeting human spleen cells.

29. Use of a recombinant AAV vector comprising an AAV3B capsid having packaged therein an expression cassette comprising an exogenous sequence encoding a gene product under control of regulatory sequences which direct expression thereof in a cell in a method for targeting human hepatocytes in a patient having pre-existing immunity to a Clade E AAV.

30. A recombinant AAV3B vector comprising an AAV3B capsid having packaged therein an expression cassette comprising an exogenous sequence encoding a gene product under control of regulatory sequences which direct expression thereof in a cell, said AAV3B vector being suitable for use in a method for targeting human hepatocytes in a patient having pre-existing immunity to a Clade E AAV.


SEQUENCE LISTING (24 sequences)

SEQ ID NO: 1 - protein, 738 aa, Homo sapiens
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 2 - protein, 6 aa, Homo sapiens
NKDKLN

SEQ ID NO: 3 - protein, 736 aa, Homo sapiens
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 736 aa, Artificial sequence
  AAVLK03 variant of AAV3
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 5 - protein, 736 aa, Artificial sequence
  variant of LK03 in which Leu at position 125 is substituted with an Ile
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 6 - protein, 736 aa, Artificial Sequence
  S663V+T492V modified AAV3B
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKVANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFVPAKFASFITQYSTGQVSVEIEWELQKENSKRWNPEIQYTSNYNKSVNVDFTVDTNGVYSEPRPIGTRYLTRPL

SEQ ID NO: 7 - DNA, 52 bp, Artificial sequence
  NQSKLN to SPAKFA primer
atcctccgaccaccttcagccctgccaagtttgcttctttcatcacgcaata

SEQ ID NO: 8 - DNA, 52 bp, Artificial sequence
  pAAV2/8 (NQSKLN to SPAKFA)
tattgcgtgatgaaagaagcaaacttggcagggctgaaggtggtcggaggat

SEQ ID NO: 9 - DNA, 52 bp, Artificial sequence
  pAAV2/rh.64R1 (NQAKLN to SPAKFA) primer
atcctccaacagcgttcagccctgccaagtttgcttctttcatcacgcagta

SEQ ID NO: 10 - DNA, 52 bp, Artificial sequence
  pAAV2/rh.64R1 (NQAKLN′SPAKFA)
tactgcgtgatgaaagaagcaaacttggcagggctgaacgctgttggaggat

SEQ ID NO: 11 - DNA, 52 bp, Artificial sequence
  pAAV2/9 (NKDKLN′SPAKFA)
atcctccaacggccttcagccctgccaagtttgcttctttcatcacccagta

SEQ ID NO: 12 - DNA, 52 bp, Artificial Sequence
  pAAV2/9 (NKDKLN to SPAKFA) Primer 2
tactgggtgatgaaagaagcaaacttggcagggctgaaggccgttggaggat

SEQ ID NO: 13 - DNA, 52 bp, Artificial sequence
  pAAV2/3B (SPAKFA to NKDKLN)
atcctccgacgactttcaacaaggacaagctgaactcatttatcactcagta

SEQ ID NO: 14 - DNA, 52 bp, Artificial Sequence
  pAAV2/3B (SPAKFA to NKDKLN) Primer 2
tactgagtgataaatgagttcagcttgtccttgttgaaagtcgtcggaggat

SEQ ID NO: 15 - protein, 6 aa, Artificial sequence
  clade D epitope
TPAKFA

SEQ ID NO: 16 - protein, 6 aa, Artificial Sequence
  Epitope
NQAKLN

SEQ ID NO: 17 - protein, 8 aa, Artificial Sequence
  AAV1 epitope
TTNDGVTT

SEQ ID NO: 18 - protein, 8 aa, Artificial Sequence
  AAV2 epitope
TQNDGTTT

SEQ ID NO: 19 - protein, 8 aa, Artificial Sequence
  AAV5 epitope
TVQDSTTT

SEQ ID NO: 20 - protein, 8 aa, Artificial Sequence
  AAVrh10 epitope
TQNEGTKT

SEQ ID NO: 21 - protein, 8 aa, Artificial Sequence
  AAVhu37 epitope
TQNEGTKT

SEQ ID NO: 22 - protein, 8 aa, Artificial Sequence
  AAVrh64R1
TQNEGTKT

SEQ ID NO: 23 - protein, 8 aa, Artificial Sequence
  AAV9 epitope
TDNNGVKT

SEQ ID NO: 24 - protein, 6 aa, Artificial Sequence
  AAV Capsid binding epitope
SPAKFA